(19) United States
(12) Patent Application Publication
(10) Pub. No.: US 2026/0137329 A1
(43) Pub. Date: May 21, 2026

(54) METHOD FOR ASSESSMENT AND CORRECTION OF THE PERSON’S FUNCTIONAL STATE AND SYSTEM FOR IMPLEMENTATION OF THIS METHOD

(71) Applicant: ZAKRYTOE AKTSIONERNOE OBSCHESTVO "EC-LEASING", Moscow (RU)

(72) Inventors: Alexander Viktorovich SHMID, Moscow (RU); Elizaveta Leonidovna SIVOKHOVA, Irkutsk (RU); Maxim Alexandrovich NOVOPASHIN, Ivanovskaya obl. (RU); Yury Konstantinovich KIRILLOV, Moscow (RU); Andrey Alexandrovich BEREZIN, Moscow (RU)

(73) Assignee: ZAKRYTOE AKTSIONERNOE OBSCHESTVO "EC-LEASING", Moscow (RU)

(21) Appl. No.: 19/393,168

(22) Filed: Nov. 18, 2025

(30) Foreign Application Priority Data
Nov. 19, 2024 (RU) RU 2024134573

(51) Int. Cl. A61B 5/346; A61B 5/00; A61B 5/01; A61B 5/0205; A61B 5/021; A61B 5/024; A61B 5/145; A61B 5/329

(52) U.S. Cl. CPC A61B 5/346; A61B 5/01; A61B 5/02055; A61B 5/021; A61B 5/02405; A61B 5/14517; A61B 5/14542; A61B 5/14546; A61B 5/329; A61B 5/4833; A61B 5/7257; A61B 5/7264

(57) ABSTRACT

A method for assessment and correction of a person's functional state involves recording electrocardiograms (ECGs) of at least the first lead. Initially, the first ECG is recorded. The second ECG is recorded after the person completes the first therapeutic test, and the third and subsequent ECGs are recorded after the second and subsequent therapeutic tests. At least one pair of ECGs is selected, and the corresponding sets of heart rate variability parameters, Vi and Vj, are calculated for each ECG from the selected pair of ECGs (ECGi and ECGj). A group of parameters Gij is formed for each calculated pair of sets, consisting of Vi, Vj, and their calculated difference, Δij. Each group Gij is processed in a functional state assessment unit using a mathematical model selected from a group consisting of a decision tree, a neural network, or one of three regressions—linear, logistic, and probit. The values of the person's functional state assessment, Eij, are obtained for each group of Gij. Each obtained value Eij is compared with the tabular assessment values selected from the table for the corresponding therapeutic test. The table comprises a set of values for assessing a functional state from 1 to M and corresponding descriptions for each assessment value of the corresponding functional state, as well as standard recommendations. The correspondence of each obtained value Eij of the person's functional state assessment to one of the assessment values provided in the table is determined. Recommendations are formed based on the descriptions of functional state and standard recommendations provided in the table for all obtained functional state assessment values for all selected pairs of recorded ECGs. The person's implementation of the formed recommendations is monitored. Adjustment is performed, if necessary. The functional state is corrected by adjusted implementation of the formed recommendations. The method allows obtaining a quality assessment value of implementation of the formed recommendations. A system for assessment and correction of the person's functional state is also proposed.

### CROSS-REFERENCE TO RELATED APPLICATION

The present application claims the priority of Russia Application No. RU 2024134573 filed Nov. 19, 2024, the disclosure of which is expressly incorporated by reference herein in its entirety.

### TECHNICAL FIELD

The present invention relates to medicine, and more specifically to a method for assessing and correcting the functional state of a person and a system for implementation the method. The method enables monitoring, assessment, and correction of a person's functional state, including before, during, and after physical activity, functional activity, respiratory activity, medicament administration, physiotherapy, psychoemotional activity, or combinations thereof, by selecting restorative, supportive, and developmental means.

The method can be used in various settings, including clinical hospitals, first-aid stations, as well as for the correction of a person's functional state outside of healthcare facilities in sports organizations, fitness clubs, gyms, resorts, schools and kindergartens, with personal support.

### BACKGROUND OF THE INVENTION

A person's functional state is a characteristic of the level of functioning of the body's systems over a given period of time, reflecting the body's adaptation to external influences. A person's physical and mental well-being, as well as the success of their work, education, and creativity, depend on the body's functional state.

Functional state is a complex of properties that determines the level of vital activity of the organism, the systemic response of the organism to the imposed load, which reflects the degree of integration and adequacy of functions to the work performed.

The following factors influence a person's functional state: properties of the nervous system; temperament type; general emotional orientation; ability to neutralize negative emotional traces; degree of development of volitional qualities; mastery of techniques for managing one's own mental state; intellectual development.

Assessing the functional state of a person, increasing the accuracy of recognizing functional states, and preventing an unacceptable decrease in the efficiency and reliability of the activities of specialists in hazardous professions is of practical interest for civil and military medicine, the field of professional selection, and systems for monitoring operator's activities.

Known from prior art is a METHOD FOR PREVENTIVE-REHABILITATION CORRECTION OF FUNCTIONAL STATE AND ENHANCEMENT OF BODY ADAPTABILITY (see, for example, RU 2557695C2, published 27 Jul. 2015). The invention refers to restorative medicine and can be used for the drug-free correction, prevention and rehabilitation of the functional state and the enhancement of adaptability in patients of various age groups suffering various psycho-neurological symptoms. There are performed three successive stages: setting (SS), adaptation (AS) and tactile contact (TCS). The SS involves introducing the patient into dolphins' organization and behavior characteristics; instructing to perform commands required for communication with a dolphin, and tactile contact with a dolphin. The adaptation stage (AS) involves meeting the living dolphin and contacting it directly on a swimming pool platform out of water. The TCS involves doing the exercises to develop patient's communicative abilities, fine motor skills, motor coordination, motor activity control, emotional state and attention with the use of art therapy elements, musical exercises together with the dolphin, and action games with the dolphin using various things, objects. The TCS is performed in the swimming pool filled with mineral water NaCl 1.8-2.0 ppm at temperature from 25 to 28 degrees; the whole correction course involves from 10 to 14 sessions, from 30 to 40 min each. The SS length is at least 3 sessions of 30 min each; the AS is from 1 to 3 sessions of 30-35 min each; the TCS includes from 7 to 9 sessions of 35-40 min each. The method provides the higher stress stability and adaptation potential of the patient of any degree of functional preparation, the integral enhancement of communicative ability and orientation to active targeted activity, the optimized length of the rehabilitation course depending on the patient's psychoemotional state and abilities.

However, this method does not provide dynamic monitoring, i.e., an assessment of a person's functional state before, during, and after a therapeutic test that includes physical activity, without interfering with physical activity. This method also does not allow for the possibility of correcting the functional state by providing precise recommendations based on the results of the examination.

Known from prior art is a method for systematizing and assessing the functional state of the body for timely correction of physical activity involved in recreational physical culture and sports and computer-implemented system for implementation the method (see, for example, RU 2808374 C1, published 28 Nov. 2023). The method refers to sports physiology and can be used in the practice of medical control to enable self-monitoring of the functional state of the body of athletes and people involved in physical education, in sports pedagogy and in the training process to assess the functional state of those involved in physical culture and sports.

The method of systematizing and assessing the functional state of the body for timely correction of physical activity involved in recreational physical culture and sports, comprises the following steps: proposing a user to select the level of functional state from the presented options based on subjective indicators. The user carries out 7 tests to collect data: counting the heart rate while lying down; counting the heart rate while standing; measuring upper blood pressure and lower blood pressure; measuring weight; conducting a test with holding the breath while inhaling—Stange probe; carrying out a test with holding the breath on exhalation—the Genche probe; carrying out the Letunov probe. Additionally the program calculates the orthostatic test as the difference between the standing heart rate and the lying heart rate.

The Stange, Genche and Letunov tests are carried out once every 14 days in the morning. Next, for objective indicators, the user enters functional state data based on the tests performed; as a result, the program automatically recalculates subjective indicators into points. From objective indicators, the program automatically calculates points for the orthostatic test, which is calculated as the difference between the standing heart rate and the lying heart rate and is converted into point values. Next, the user performs Letunov's tests; after which the program displays all the results obtained on the monitor screen of a computer-implemented system, builds graphs, and recalculates the results in the form of values with post-load changes, based on the results of which the type of response to the load is determined. Next, the functional state is further assessed based on the obtained indicators. Said invention provides the ability to self-monitor changes in the state of the user's functional parameters in real time, and allows to perform recording, accumulation, storage and evaluation of objective and subjective indicators of the user's functional state to analyze the degree of readiness of the user for the possibility of correcting physical activity if necessary.

This method has a drawback—a long period of time for collecting samples, but the functional state of a person over such a long period of time can be changed significantly, as a result of which an objective assessment of the functional state at the current moment in time is not ensured.

However, this method does not provide dynamic monitoring, i.e., an assessment of a person's functional state before, during, and after a therapeutic test including physical activity and stress, without interfering with physical activity. This method also does not allow for the possibility of correcting of functional state by providing precise recommendations based on the results of the examination.

The closest analog to the claimed technical solution is a method of pre-medical assessment of a person's functional state and automatic system for assessment functional state of user (versions) (see RU 2445916 C2, published 27 Mar. 2012).

The method comprises: biological signal reading by at least one sensor; recording; automatic analysis, including calculation of the variability of cardiac rhythm and index of activity of regulatory systems; extraction of the parameters specifying a risk of cardiac disturbances, cardiac arrest emergency, and also diagnosed cardiac rhythm disorder; and functional state data display. Additionally, personal information on current patient's state as of the time of biological signal reading is recorded; probable reasons of the functional state variation in accordance with pre-recorded history, current health and parameters exceeding the threshold values of normal ECG or parameters of previous ECGs are listed; the functional state assessment is expressed in the form to be presented to an individual being examined.

A system for implementing the method comprises biological signal sensors, a recorder and an analysis unit for recorded biological signals. Further, the system is provided with a user terminal to input health data, a unit for automatic listing of probable reasons of the functional state variation and functional state assessment to be presented to an individual, connected with the analysis unit for recorded biological signals. A second embodiment of the system is characterized by the fact it comprises the user terminal to input health data from the biological signal recorder or current health values.

The method does not provide dynamic monitoring, i.e., an assessment of a person's functional state before, during, and after a therapeutic test that includes physical activity and stress, without interfering with physical activity. The method assesses functional state by recording an electrocardiogram, but without taking into account the dynamics of changes in these parameters, which reduces the reliability of the assessment. The method does not allow reducing the time required to conduct a functional assessment for an individual or group of individuals, as it requires additional recording of the individual's medical history and current well-being. The functional assessment results in this method are determined by parameters that exceed the electrocardiogram thresholds corresponding to normal functioning or parameters from previous electrocardiograms. This makes it impossible to present the functional assessment results in tabular form, using language accessible to individuals without specialized medical training. This means that recommendations cannot be formed for a patient based on the text descriptions of their functional state found in the table, nor can monitoring of their implementation be ensured. This method does not allow for the possibility of correcting the functional state by providing precise, individualized recommendations based on the results of the assessment.

### SUMMARY OF THE INVENTION

The object of the claimed invention is to provide a method for assessing and correcting a person's functional state, in which the functional state of a person is assessed based on changes in the parameters of heart rate variability (HRV) or based on changes in the discretized electrocardiogram (ECG) after subjecting the person to at least one therapeutic test, thereby ensuring an increase in the reliability of the result of assessing the functional state of a person and a reduction in the duration of the assessment, and, based on the obtained assessment of the functional state, forming recommendations for conducting a training load, a therapeutic load, medicament administration and monitoring the implementation of the formed recommendations and timely correction of their implementation, thereby ensuring the correction of the management of the functional state of a person based on precise recommendations based on the results of the examination.

The second object of the present invention is to provide a system for assessing and correcting a person's functional state, which ensures the implementation of the specified method.

According to the first embodiment of the invention, the stated object is achieved by creating a method for controlling the assessment and correction of a person's functional state, the method comprising the following steps:
recording two electrocardiograms (ECGs) of at least a first lead, wherein the first ECG1 is recorded initially, and the second ECG2 is recorded after the person has completed a therapeutic test;
calculating corresponding sets of heart rate variability (HRV) parameters V1 and V2 for the recorded ECG1 and ECG2;
calculating a difference Δ between the obtained sets of heart rate variability parameters V1 and V2;
forming a group G of parameters consisting of the set of HRV parameters V1, the set of HRV parameters V2, and the calculated difference Δ between the sets of parameters;
processing the formed group G of parameters consisting of V1, V2, and A in a unit for assessing the person's functional state using a mathematical model; said mathematical model is selected from a group consisting of a decision tree, a neural network, and one of three regressions: linear, logistic, and probit, to obtain the value E of the person's functional state assessment;
comparing the obtained value of the person's functional state assessment with the tabular assessment values selected from the table for the corresponding therapeutic test, wherein the table for the corresponding therapeutic test comprises a set of functional state assessment values from 1 to M and descriptions for each assessment value of the corresponding functional state, as well as standard recommendations; and identifying the compliance of the obtained functional state assessment value to one of the assessment values provided in the table of the corresponding therapeutic test;
forming recommendations for the person based on the functional state descriptions and standard recommendations provided in the table for the identified functional state assessment value;
monitoring the implementation of the formed recommendations and adjusting the implementation process based on the monitoring data;
implementing the correction of the functional state by following the formed recommendations by the person.

Preferably, the step of monitoring the implementation of formed recommendations is performed by taking at least one measurement selected from the group consisting of heart rate, palpation, percussion, auscultation, blood pressure, temperature, blood saturation, blood lactate accumulation level, spirometry, and the Minor test during the implementation of recommendations, or by recording cardiographic data or biometric data during the implementation of recommendations.

Preferably, the therapeutic test comprises at least one exercise selected from the group consisting of physical exercise, functional load tests, respiratory exercise, physiotherapeutic exercise, psychoemotional practice, and/or at least one influence selected from the group consisting of medicament administration, physiotherapeutic influence, psychoemotional influence, respiratory influence, or combinations thereof.

Preferably, the descriptions in the table for functional state assessment value for each corresponding functional state are selected from the group consisting of text explanations, videos, images, video instructions, diagrams, animations, or combinations thereof.

Preferably, performing the step of identification of the compliance of the obtained person's functional state assessment value to one of the assessment values from 1 to M=6, provided in the table and including six states.

Preferably, when using the therapeutic test from the group consisting of physical exercise, functional load test, physiotherapeutic exercise, and psychoemotional practice, or combinations thereof,
providing an identification of compliance of the obtained person's functional state assessment value to one of the assessment values from 1 to M=6, provided in the table and comprising six states selected from the group consisting of critical overstrain, overstrain, fatigue, incomplete recovery, complete recovery, and supercompensation.

Preferably, when using respiratory exercise,
an identification of compliance of the obtained person's functional state assessment value to one of the assessment values from 1 to M=6, provided in the table and comprising six states selected from the group consisting of a pathological state, overstrain of the gas exchange systems, insufficient compensation of gas exchange, moderate compensation of gas exchange, good compensation of gas exchange, and supercompensation is performed.

Preferably, the method comprises the step of identification of compliance of the obtained person's functional state assessment value to one of the assessment values from 1 to M=5, given in the table and including five states.

Preferably, when using the therapeutic test from the group consisting of medicament administration, physiotherapeutic influence, psychoemotional influence, respiratory influence, or combinations thereof,
an identification of compliance of the obtained person's functional state assessment value to one of the assessment values from 1 to M=5, provided in the table and comprising five states selected from the group consisting of favorable improvement, unfavorable improvement, favorable deterioration, unfavorable deterioration, and unchanged state is performed.

Preferably, the method comprises the step of identification of compliance of the obtained person's functional state assessment value to one of the assessment values from 1 to M=4, provided in the table and including four states.

Preferably, the method comprises the step of performing an identification of compliance of the obtained person's functional state assessment value to one of the assessment values from 1 to M=4, provided in the table and comprising four states selected from the group consisting of overstrain, fatigue, recovery, and supercompensation.

Preferably, further a step is performed of obtaining the person's functional state assessment values in the range from 0 to M=10, wherein the value 0 corresponds to the worst functional state of a person, and the value 10 corresponds to the best functional state of a person.

According to the second embodiment of the invention, the stated object is achieved by creating a method for assessment and correction of a person's functional state, the method comprising the steps of:
recording at least three electrocardiograms (ECGs) of at least a first lead, wherein the first ECG1 is recorded initially, the second ECG2 is recorded after the person has completed a therapeutic test, and the third ECG3 and subsequent ECGs are recorded after the person has completed the second and subsequent therapeutic tests, respectively;
selecting at least one pair of recorded electrocardiograms ECGi and ECGj, where said indices i and j are selected from the range from 1 to N, where N is the number of recorded ECGs, wherein i≠j;
calculating corresponding sets Vi and Vj of heart rate variability (HRV) parameters for each ECG from the selected at least one pair of ECGi and ECGj;
calculating a difference Δij between each calculated at least one pair of sets Vi and Vj of the HRV parameters;
forming a group Gij of parameters for each calculated at least one pair of sets of Vi and Vj of the HRV parameters, consisting of the set of Vi of the HRV parameters, the set of Vj of the HRV parameters, and their calculated difference Δij,
processing each of the formed group Gij of parameters in a unit for assessing the person's functional state using a mathematical model; said mathematical model is selected from a group consisting of a decision tree, a neural network, and one of three regressions: linear, logistic, and probit, to obtain the value Eij of the person's functional state assessment for each formed group Gij of parameters;
comparing each obtained person's functional state assessment value Eij of the corresponding selected pair of recorded ECGi and ECGj with the tabular assessment values selected from the table for the corresponding therapeutic test, wherein the table for the corresponding therapeutic test comprises a set of functional state assessment values from 1 to M and descriptions for each assessment value of the corresponding functional state, as well as standard recommendations; and identifying the compliance of the obtained functional state assessment value Eij to one of the assessment values provided in the table of the corresponding therapeutic test;
forming recommendations for the person based on the functional state descriptions and standard recommendations provided in the table for the identified functional state assessment value for all pairs of recorded ECGs;
monitoring the implementation of the formed recommendations and adjusting the implementation process based on the monitoring data;
implementing the correction of the functional state by following the formed recommendations by the person.

Preferably, the step of monitoring the implementation of formed recommendations is performed by taking at least one measurement selected from the group consisting of heart rate, palpation, percussion, auscultation, blood pressure, temperature, blood saturation, blood lactate accumulation level, spirometry, and the Minor test during the implementation of recommendations, or by recording cardiographic data or biometric data during the implementation of recommendations.

Preferably, the therapeutic test comprise at least one exercise selected from the group consisting of physical exercise, functional load tests, respiratory exercise, physiotherapeutic exercise, psychoemotional practice, and/or at least one influence selected from the group consisting of medicament administration, physiotherapeutic influence, psychoemotional influence, respiratory influence, or combinations thereof.

Preferably, the descriptions in the table for functional state assessment value for each corresponding functional state are selected from the group consisting of text explanations, videos, images, video instructions, diagrams, animations, or combinations thereof.

Preferably, the method comprises the step of identification of compliance of the obtained person's functional state assessment value to one of the assessment values from 1 to M=6, provided in the table and including six states.

Preferably, when using the therapeutic test from the group consisting of physical exercise, functional load test, physiotherapeutic exercise, and psychoemotional practice, or combinations thereof,
an identification of compliance of the obtained person's functional state assessment value to one of the assessment values from 1 to M=6, provided in the table and comprising six states selected from the group consisting of critical overstrain, overstrain, fatigue, incomplete recovery, complete recovery, and supercompensation is performed.

Preferably, when using respiratory exercise,
an identification of compliance of the obtained person's functional state assessment value to one of the assessment values from 1 to M=6, provided in the table and comprising six states selected from the group consisting of a pathological state, overstrain of the gas exchange systems, insufficient compensation of gas exchange, moderate compensation of gas exchange, good compensation of gas exchange, and supercompensation is performed.

Preferably, the method comprises the step of identification of compliance of the obtained person's functional state assessment value to one of the assessment values from 1 to M=5, provided in the table and comprising five states.

Preferably, when using the therapeutic test from the group consisting of medicament administration, physiotherapeutic influence, psychoemotional influence, respiratory influence, or combinations thereof,
an identification of compliance of the obtained person's functional state assessment value to one of the assessment values from 1 to M=5, provided in the table and comprising five states selected from the group consisting of favorable improvement, unfavorable improvement, favorable deterioration, unfavorable deterioration, and unchanged state is performed.

Preferably, the method comprises the step of identification of compliance of the obtained person's functional state assessment value to one of the assessment values from 1 to M=4, provided in the table and comprising four states.

Preferably, the method comprises the step of identifying the compliance of the obtained person's functional state assessment value to one of the assessment values from 1 to M=4, provided in the table and comprising four states selected from the group consisting of overstrain, fatigue, recovery, and supercompensation.

Preferably, the step of obtaining the person's functional state assessment values in the range from 0 to M=10, wherein the value 0 corresponds to the worst functional state of a person, and the value 10 corresponds to the best functional state of a person is further performed.

Preferably, the method further comprises a step of obtaining a quality assessing value of implementation of the formed recommendations, by performing the following steps:
recording at least two electrocardiograms (ECGs) of at least a first lead; calculating corresponding heart rate variability (HRV) parameters; calculating the difference Δ between the HRV parameter; forming and processing a group G of parameters; and obtaining a person's functional state assessment value E before implementation of recommendations; then forming and implementing recommendations by performing the method steps of claim 14;
recording at least one additional ECG, wherein a therapeutic test is performed by the person between each successive pair of additional ECGs;
processing the additional ECGs and obtaining a person's functional state assessment value after implementation of recommendations;
comparing the obtained value before receiving the recommendations and the value after implementation of recommendations with the values provided in a table of quality assessment values to obtain the quality assessment value of implementation of recommendations, wherein said table of quality assessment values comprises the assessment values before performing the recommendations, the assessment values after implementation of recommendations, and the corresponding quality assessment values of implementation of recommendations.

Preferably, the step of identification of compliance of the obtained quality assessment value of implementation of recommendations and the table descriptions of quality assessment values of implementation of recommendations is further performed, wherein the table descriptions of quality assessment values of implementation of recommendations comprises a set of quality assessment values of implementation of recommendations from 1 to Q and corresponding descriptions for each assessment value.

Preferably, the descriptions in the table descriptions of quality assessment values of implementation of recommendations are selected from the group consisting of text explanations, videos, images, video instructions, diagrams, animations, or combinations thereof.

According to the third embodiment of the invention, the stated object is achieved by a method for assessment and correction of a person's functional state, the method comprising the steps of:
recording at least two electrocardiograms (ECGs) of at least a first lead, wherein the first ECG1 is recorded initially, the second ECG2 is recorded after the person has completed a therapeutic test, and each subsequent ECGs is recorded after the person has completed subsequent therapeutic tests;
selecting at least one pair of recorded electrocardiograms ECGi and ECGj, where said indices i and j are selected from the range from 1 to N, where N is the number of recorded ECGs, wherein i≠j;
performing a Fourier transform for each ECG from the selected at least one pair of ECGi and ECGj to obtain a Fourier amplitude spectrum for each recorded ECG, and discretizing each obtained Fourier amplitude spectrum to obtain a discrete form of the Fourier amplitude spectrum (DFECG) for each ECG from the selected at least one pair of ECGi and ECGj;
calculating a difference Δ DFECGij between the discrete types of the Fourier amplitude spectrum DFECGi and DFECGj for each selected at least one pair of ECGi and ECGj;
forming a group Gij of parameters for each selected at least one pair of sets of Vi and Vj of the HRV parameters, consisting of the set of Vi of the HRV parameters, the set of Vj of the HRV parameters, and their calculated difference Δ DFECGij;
processing the formed group Gij of parameters in a unit for assessing the person's functional state using a mathematical model; said mathematical model is selected from a group consisting of a decision tree, a neural network, and one of three regressions: linear, logistic, and probit, to obtain the person's functional state assessment value Eij for each formed group Gij;
comparing each obtained person's functional state assessment value Eij of the corresponding selected pair of recorded ECGi and ECGj with the tabular assessment values selected from the table for the corresponding therapeutic test, wherein the table for the corresponding therapeutic test comprises a set of functional state assessment values from 1 to M and descriptions for each assessment value of the corresponding functional state, as well as standard recommendations; and identifying the compliance of the obtained functional state assessment value Eij to one of the assessment values provided in the table of the corresponding therapeutic test;
forming recommendations for the person based on the functional state descriptions and standard recommendations provided in the table to identified functional state assessment value for all selected pairs of the recorded ECGs;
monitoring the implementation of the formed recommendations and adjusting the implementation process based on the monitoring data;
implementing the correction of the functional state by following the formed recommendations by the person.

Preferably, the step of monitoring the implementation of formed recommendations is performed by taking at least one measurement selected from the group consisting of heart rate, palpation, percussion, auscultation, blood pressure, temperature, blood saturation, blood lactate accumulation level, spirometry, and the Minor test during the implementation of recommendations, or by recording cardiographic data or biometric data during the implementation of recommendations.

Preferably, the therapeutic tests comprise at least one exercise selected from the group consisting of physical exercise, functional load tests, respiratory exercise, physiotherapeutic exercise, psychoemotional practice, and/or at least one influence selected from the group consisting of medicament administration, physiotherapeutic influence, psychoemotional influence, respiratory influence, or combinations thereof.

Preferably, the descriptions in the table for functional state assessment value for each corresponding functional state are selected from the group consisting of text explanations, videos, images, video instructions, diagrams, animations, or combinations thereof.

Preferably, the method comprises the step of identification of compliance of the obtained person's functional state assessment value to one of the assessment values from 1 to M=6, provided in the table and including six states.

Preferably, when using the therapeutic test from the group consisting of physical exercise, functional load test, physiotherapeutic exercise, and psychoemotional practice, or combinations thereof,
an identification of compliance of the obtained person's functional state assessment value to one of the assessment values from 1 to M=6, provided in the table and comprising six states selected from the group consisting of critical overstrain, overstrain, fatigue, incomplete recovery, complete recovery, and supercompensation is performed.

Preferably, when using respiratory exercise,
an identification of compliance of the obtained person's functional state assessment value to one of the assessment values from 1 to M=6, provided in the table and comprising six states selected from the group consisting of a pathological state, overstrain of the gas exchange systems, insufficient compensation of gas exchange, moderate compensation of gas exchange, good compensation of gas exchange, and supercompensation is performed.

Preferably, the method comprises the step of identification of compliance of the obtained person's functional state assessment value to one of the assessment values from 1 to M=5, provided in the table and comprising five states.

Preferably, when using the therapeutic test from the group consisting of medicament administration, physiotherapeutic influence, psychoemotional influence, respiratory influence, or combinations thereof,
an identification of compliance of the obtained person's functional state assessment value to one of the assessment values from 1 to M=5, provided in the table and comprising five states selected from the group consisting of favorable improvement, unfavorable improvement, favorable deterioration, unfavorable deterioration, and unchanged state is performed.

Preferably, the method comprises the step of identification of compliance of the obtained person's functional state assessment value to one of the assessment values from 1 to M=4, provided in the table and comprising four states.

Preferably, the method comprises the step of identification of compliance of the obtained person's functional state assessment value to one of the assessment values from 1 to M=4 provided in the table and comprising four states selected from the group consisting of overstrain, fatigue, recovery, and supercompensation.

Preferably, the method comprises a step of obtaining the person's functional state assessment values in the range from 0 to M=10, wherein the value 0 corresponds to the worst functional state of a person, and the value 10 corresponds to the best functional state of a person.

Preferably, the method further comprises a step of obtaining a quality assessment value of implementation of the formed recommendations, by performing the following steps:
recording at least two electrocardiograms (ECGs) of at least a first lead; performing a Fourier transform for each ECG; discretizing each obtained Fourier amplitude spectrum to obtain a discrete form of the Fourier amplitude spectrum (DFECG) for each ECG; calculating the difference Δ between the obtained DFECG; forming and processing a group G of parameters and obtaining a person's functional state assessment value E before implementation of recommendations;
forming and implementing recommendations by the method steps of claim 14;
recording at least one additional ECG, wherein a therapeutic test is performed by the person between each successive pair of additional ECGs;
processing the additional ECGs and obtaining a person's functional state assessment value after implementation of recommendations;
comparing the obtained person's functional state assessment value before receiving the recommendations and the value after implementation of recommendations with the values provided in a table of quality assessment values to obtain the quality assessment value of implementation of recommendations, wherein said table of quality assessment values comprises the assessment values before performing the recommendations, the assessment values after implementation of recommendations, and the corresponding quality assessment value of implementation of recommendations.

Preferably, the method further comprises a step of identifying the compliance of the obtained quality assessment value of implementation of recommendation with the table descriptions of quality assessment values of implementation of recommendation, wherein the table descriptions of quality assessment values of implementation of recommendation comprises a set of quality assessment values from 1 to Q and corresponding descriptions for each assessment value.

Preferably, the descriptions in the table descriptions of quality assessment values of implementation of recommendation are selected from the group consisting of text explanations, videos, images, video instructions, diagrams, animations, or combinations thereof.

According to the fourth embodiment of the invention, the stated object is achieved by creating a system for assessment and correction of a person's functional state, comprising:

an examination scenario control unit, connected to an electrocardiograph configured to record a series of electrocardiograms (ECG) of at least a first lead before and after the person has fulfilled at least one therapeutic test, and connected to an ECG accumulation unit, which in turn is connected to the electrocardiograph, and connected in series:
a unit for calculation sets of characteristic parameters for each of the recorded ECGs, connected to the ECG accumulation unit;
a unit for calculation a difference Δ between pairs of the sets of characteristic parameters and for forming a group G of parameters, said group G consists of two sets of characteristic parameters and the calculated difference Δ;
a unit for assessment a functional state of a person, configured to process the formed group of parameters and obtain a person's functional state assessment value by using a mathematical model selected from a group consisting of a decision tree, a neural network, one of three regressions—linear, logistic, and probit;
a unit for selection a description of a functional state and standard recommendations from a built-in table for corresponding therapeutic test, in accordance with the obtained assessment value, wherein the table comprises a set of functional state assessment values from 1 to M and descriptions of the corresponding functional state and standard recommendations for each assessment value;
a unit forming and storing connected to the selection unit, the forming and storing unit being configured for forming and storing recommendations;
a unit for accumulating monitoring data, configured for accumulation data being the results of monitoring the process of fulfilment of the formed recommendations, and further for detecting deviations from implementation of recommendations;
a unit for forming instructions for correction the process of implementing the formed recommendations for correction the person's functional state.

Preferably the system further comprises a unit for inputting monitoring data, connected to the unit for accumulating monitoring data.

Preferably the system further comprises a unit for updating formed recommendations, connected to the unit for forming and storing recommendations.

Preferably the unit for calculation sets of characteristic parameters for each of the recorded ECGs is configured for calculating a set of heart rate variability parameters.

Preferably the cardiograph is configured for determining the moments of heart contractions.

Preferably the unit for calculation sets of characteristic parameters is configured for determining the moments of heart contractions.

Preferably the unit for calculation sets of characteristic parameters is configured to calculate a discretized ECG (DFECG).

The technical result of the claimed invention is as follows. The claimed method provides increasing the reliability of the results of person's functional state assessment value and expanding the range of automated systems for person's functional state assessment value. It is possible to reduce the time required for conducting a reliable assessment of a person's functional state, thereby making it possible to assess a person's functional state in a shorter period compared to clinical assessments of functional capabilities. The method makes it possible to present the results of assessing a person's functional state in a tabular form accessible to individuals without a specialized medical training. Additionally, it becomes possible to early detection of disorders of a person's practical health. Most importantly, the method provides the ability to correct a person's functional state based on precise recommendations based on the results of the examination.

### BRIEF DESCRIPTION OF DRAWINGS

The invention is hereinafter explained by the description of preferred embodiments thereof with reference to the accompanying drawings, in which:

FIG. 1 shows an algorithm for assessing a person's functional state value, according to the first embodiment of the invention;

FIG. 2 shows an algorithm for assessing a person's functional state value, according to the second embodiment of the invention;

FIG. 3 shows an algorithm for assessing a person's functional state value and a value for assessing the quality of implementation of the formed recommendations, according to the second embodiment of the invention (recording three ECGs);

FIG. 4 shows an algorithm for assessing a person's functional state value and a value for assessing the quality of implementation of the formed recommendations, according to the second embodiment of the invention (recording four ECGs);

FIG. 5 shows an algorithm for assessing a person's functional state value, according to the third embodiment of the invention;

FIG. 6 shows an algorithm for assessing a person's functional state value and a value for assessing the quality of implementation of the formed recommendations, according to the second embodiment of the invention (recording three ECGs);

FIG. 7 shows a block-diagram of a system for assessment and correction of a person's functional state value.

### DESCRIPTION OF PREFERRED EMBODIMENTS

Until now, no one has assessed a person's functional state based on changes in heart rate variability (HRV) parameters or based on changes of discrete form of the electrocardiogram (ECG) after subjecting the person to at least one therapeutic test.

Unexpectedly, it was revealed that a reliability of a person's functional state assessment results significantly increases and the time required for the assessment is reduced if a person's functional state is evaluated based on changes in HRV parameters after subjecting the person to at least one therapeutic test, since the heart rhythm comprises information about the functional state of all systems regulating human life activity, both normally and in various pathologies.

The method for assessment and correction of a person's functional state, according to the first embodiment, is implemented as follows.

It is proposed to consider the implementation of the method using the following example of for assessment and correction of a person's functional state.

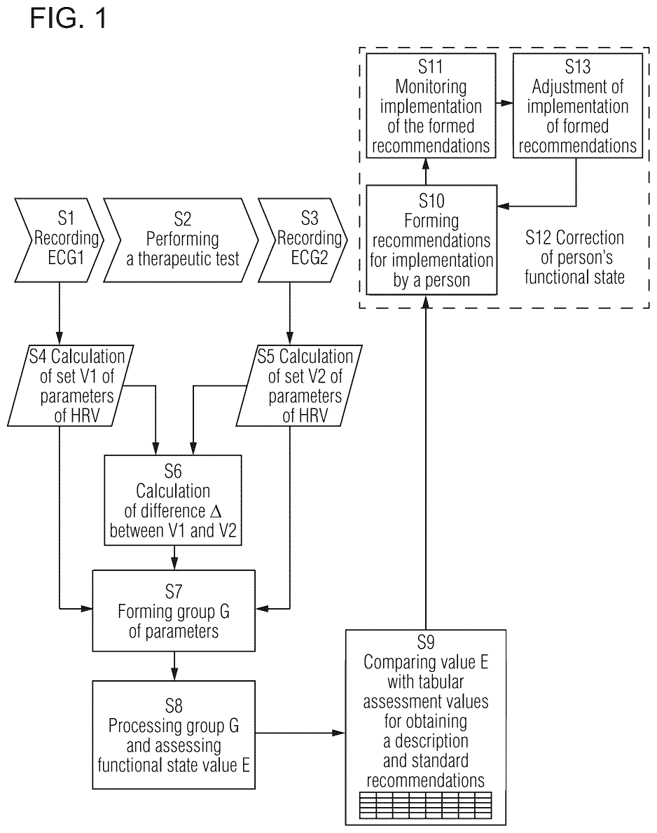

Two electrocardiograms (ECGs) of at least a first lead are recorded (FIG. 1), wherein the first ECG1 is recorded S1 initially, the person has completed S2 a therapeutic test and then the second ECG2 is recorded S3.

In the described embodiment, the term “therapeutic test” means either an exercise performed by a person, or an influence to which a person is subjected. As it is indicated above the therapeutic test comprises at least one exercise selected from the group consisting of physical exercise, functional load tests, respiratory exercise, physiotherapeutic exercise, psychoemotional practice, and/or at least one influence selected from the group consisting of medicament administration, physiotherapeutic influence, psychoemotional influence, respiratory influence, or combinations thereof.

The therapeutic test may comprise at least one exercise and/or at least one influence.

The exercise is performed according to a strict schedule, such as 30 arm swings in 45 seconds or 20 squats in 45 seconds. The time is limited to 45 seconds, whereas the number of exercises and the type of exercises may vary.

The influence is performed without a strict schedule, and comprises, for example, an intravenous medicament administration or eating.

Physical activity comprises the following: squats with a specified frequency, the number of squats is determined by the person's level of functional and physical capabilities; arm swings with a specified frequency, the number of swings is determined by the person's level of functional and physical capabilities; push-ups with a specified frequency, the number of push-ups is determined by the person's level of functional and physical capabilities; a training exercises or a regulated load test on an ergometer; and active and passive rest, as determined by the examination schedule.

If there are no functional or physical limitations, the prescribed physical exercise is 30 squats for 45 seconds.

If functional or physical limitations exist, 10 or 20 squats for 45 seconds are used, or they are replaced with 10, 20, or 30 high-amplitude arm swings for 45 seconds. If squats are impossible or the lower limbs are dysfunctional, squats can be replaced with 10, 20, or 30 push-ups from the floor or from the wheelchair handles for 45 seconds, depending on the limitations. If the person has excellent athletic ability and is in excellent physical state, squats can be replaced with 30 push-ups for 45 seconds.

When analyzing the training process, training sessions and exercises themselves are considered as a physical exercise, as well as active and passive rest, as determined by the examination protocol.

When analyzing the person's athletic form, a regulated load test on an ergometer is used as a physical exercise, for example, a bicycle ergometer, a treadmill, a hand ergometer, a rowing ergometer, a ski ergometer, etc.

The most common functional load tests include spirometry, orthostatic probe, body plethysmography, carbon dioxide diffusion capacity testing using a single breath-hold method, stress testing to detect bronchial hyper reactivity, drinking 200-250 ml of clean water, glucose tolerance testing, the Stange probe, the Genche probe, the Letunov probe, and other less common tests.

Physiological influences include urination, defecation, vomiting, expectoration, nose blowing, and sexual intercourse.

Respiratory exercises include forced inhalations and slow exhalations at a regulated frequency, the number of which is determined by the functional and physical capabilities of the respiratory and auxiliary systems.

If there are no limitations on the functional and physical capabilities of the respiratory and auxiliary systems, 30 forced inhalations and slow exhalations over 45 seconds are used as a regulated respiratory exercise.

If the functional and physical capabilities of the respiratory and auxiliary systems are limited, 10-20 forced inhalations and slow exhalations over 45 seconds can be used, or replaced with one or more breath holds of up to 45 seconds.

Physiotherapeutic exercises include specialized exercises in cryotherapy, heat therapy, hydrotherapy, and barotherapy.

Psycho-emotional influences include psychological pressure and stress experienced during work, sports, or social activities.

The following physiotherapeutic influences can be performed: electrotherapy, shock wave therapy, magnetic therapy, laser therapy, ultrasound therapy, infrared irradiation, cryotherapy, heat therapy, hydrotherapy, barotherapy, phonophoresis, electropuncture, amplipulse therapy, myostimulation, electromagnetic therapy, including complex therapy using an FPU generator (disclosed in patent RU 2757254).

Psychoemotional practices include persuasion, the spread of ideas, gossip, or habits, suggestion, imitation, fashion, attending entertainment events, listening to music, and watching videos.

Respiratory influences include cardiorespiratory testing, oxygen saturation or intoxication, carbon dioxide saturation, and the use of devices to increase or decrease air pressure during inhalation and/or exhalation.

Then, the sets of V1 and V2 heart rate variability (HRV) parameters are calculated S4 and S5 for each of the two recorded ECG1 and ECG2, respectively, including the geometric, statistical, and spectral parameters of HRV.

The geometric parameters represent the distribution of RR intervals as random value, while the distribution curve of RR intervals is a histogram. Statistical parameters represent a direct quantitative assessment of HRV during the studied period; a cardiointervalogram is considered as a sequence of RR intervals. Spectral parameters represent a quantitative assessment of various frequency components of heart rate oscillations and a graphical representation of ratios of different heart rate components, reflecting the activity of certain links in a regulatory mechanism.

A difference Δ between the corresponding parameters of the obtained sets V1 and V2 of heart rate variability parameters is calculated S6; and group G of parameters is formed S7. Group G consists of a set V1 of HRV parameters, a set V2 of HRV parameters and the calculated difference Δ between the sets of parameters.

The difference Δ between the sets of HRV parameters is a set of indicators, each of which is a value calculated using a formula selected from the group consisting of:
a difference between parameter p (i) of set V1 and parameter p (i) of set V2;
a ratio of parameter p (i) of set V1 to parameter p (i) of set V2;
a ratio of the difference between parameter p (i) of set V1 and parameter p (i) of set V2 to the original value of parameter p (i), expressed as a percentage;
the ratio of the difference between parameter p (i) of set V1 and the accepted medical norm for this parameter to the difference between parameter p (i) of set V2 and the accepted medical norm for this parameter;
the ratio of the absolute value of the difference between parameter p (i) of set V1 and the accepted medical norm for this parameter to the absolute value of the difference between parameter p (i) of set V2 and the accepted medical norm for this parameter;
the ratio of the difference between parameter p (i) and parameter p (j) of set V1 to the difference between parameter p (i) and parameter p (j) of set V2;
the ratio of the absolute value of the difference between parameter p (i) and parameter p (j) of set V1 to the absolute value of the difference between parameter p (i) and parameter p (j) of set V2.

The formed group G of parameters consisting of V1, V2, and A is processed S8 in a unit for assessing the person's functional state value E using a mathematical model. Said mathematical model is selected from a group consisting of a decision tree, a neural network, and one of three regressions: linear, logistic, and probit, to obtain the value E of the person's functional state assessment.

The obtained person's functional state assessment value E is compared (S9) with the tabular assessment values selected from the table for the corresponding therapeutic test. The table for the corresponding therapeutic test comprises a set of functional state assessment values from 1 to M and descriptions for each assessment value of the corresponding functional state, as well as standard recommendations. The compliance of the obtained functional state assessment value to one of the assessment values provided in the table for the corresponding therapeutic test is identified.

A table was drawn up in advance through a practical study of groups of persons (e.g., a group of athletes, a group of persons undergoing cardiac rehabilitation, or a group of persons involved in physical training). Further analysis of the collected datasets of heart rate variability parameters was performed using expert assessment methods. Various tables comprising descriptions and recommendations for different functional state assessment values were drawn up; said recommendations represent standard recommendations. Different tables were drawn up for different applications.

Practical research includes: recording ECGs before and after a therapeutic test; assessing the recorded ECGs; and forming a mathematical model using expert assessment method for a set of identified standard functional states revealed for a group of examined persons.

A corresponding table was drawn up for each group of identified standard functional states of the persons.

Generally, six states were proposed for consideration, however five states were considered in certain application areas when providing a therapeutic test, or four states when assessing a complex therapeutic effect, for example an influence combined with an exercise.

The expert assessment method is an examination method conducted by physicians and processing the opinions of the physicians who performed evaluation manually based on the data collected during examination of persons.

Each identified standard functional state was assessed, a description of this state was provided, and standard recommendations for improving or maintaining this functional state of a person were described.

According to the claimed method, recommendations are formed S10 for a person for implementation, said recommendations are based on descriptions of functional state and standard recommendations for the identified functional state assessment value provided in the table.

The implementation of the formed recommendations are monitored S11 and the obtained monitoring data is stored. Implementation of the formed recommendations is adjusted based on the monitoring data.

The person's functional state is corrected S12 by implementation of the formed recommendations and an adjustment of it based on the monitoring data. The adjustment of implementation of formed recommendations is described in the examples below.

Descriptions in the table for each assessment value of the corresponding functional state are selected from a group consisting of text explanations, videos, images, video instructions, diagrams, animations, or combinations thereof, which is very convenient for both the person and the doctor.

When using an exercise from a group consisting of physical exercise, functional load tests, respiratory exercise, physiotherapeutic exercise, psychoemotional practice the obtained assessment value of the person's functional state is determined to correspond to one of the assessment values from 1 to M=6, given in the table and corresponding to six states selected from the group consisting of critical overstrain, overstrain, fatigue, incomplete recovery, complete recovery, and supercompensation.

When using respiratory exercise, an identification of compliance of the obtained person's functional state assessment value to one of the assessment values from 1 to M=6 provided in the table, is performed. Six states are described in the table, selected from the group consisting of a pathological state, overstrain of the gas exchange systems, insufficient compensation of gas exchange, moderate compensation of gas exchange, good compensation of gas exchange, and supercompensation.

When using an influence from the group consisting of medicament administration, physiotherapeutic influence, psychoemotional influence, respiratory influence, or combinations thereof, an identification of compliance of the obtained value of the person's functional state assessment to one of the assessment values from 1 to M=5, provided in the table is performed. Five states are described in the table, selected from the group consisting of favorable improvement, unfavorable improvement, favorable deterioration, unfavorable deterioration, and unchanged state.

According to other embodiment, the method comprises the step of identification of compliance of the obtained person's functional state assessment value to one of the assessment values from 1 to M=4, provided in the table and comprising four states selected from the group consisting of overstrain, fatigue, recovery, and supercompensation.

A possible embodiment involves using a mathematical model to obtain a person's functional state assessment value in the range from 0 to 10. Within this range, a value of 0 corresponds to the person's worst functional state, and a value of 10 corresponds to the person's best functional state.

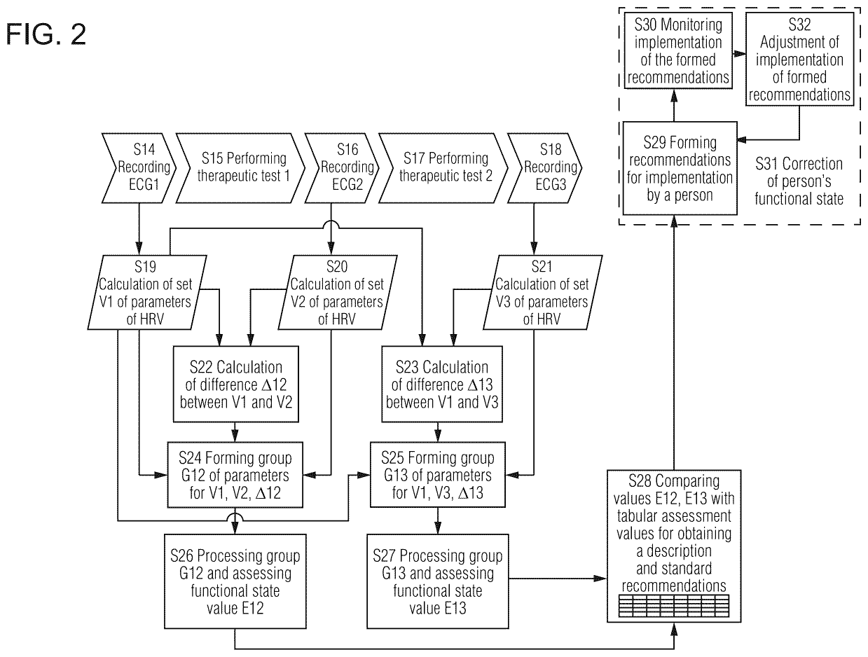

The second embodiment of the method differs from the first embodiment in that multiple electrocardiograms are recorded. FIG. 2 shows an embodiment of recording S14, S16, S18 of three electrocardiograms (ECG1, ECG2, and ECG of at least the first lead), wherein the first ECG1 is recorded first, the second ECG2 is recorded after the person has completed S15 the first therapeutic test, and the third ECG3 is recorded after the person has completed S17 the second therapeutic test, respectively. Possible therapeutic tests are described above.

The method according to the second embodiment is implemented as follows.

At least one pair of recorded ECGi and ECGj are selected, and for each at least one selected pair of ECGi and ECGj sets Vi and Vj of heart rate variability (HRV) parameters are calculated.

In the described embodiment, two pairs of recorded ECGs are selected, for example, ECG1 and ECG2 and ECG1 and ECG3. For each ECG from the recorded ECG1, ECG2, and ECG3, the corresponding sets of heart rate variability parameters V1, V2, and V3 are calculated S19, S20, S21.

The difference Δij between each at least one calculated pair of sets of HRV parameters Vi and Vj is calculated, and a group of parameters Gij is formed for each at least one calculated pair of sets of HRV parameters Vi and Vj, consisting of the set of HRV parameters Vi, the set of HRV parameters Vj, and their calculated difference Δij.

In this embodiment, two differences Δ12 and Δ13 are calculated S22 and S23 for each specified pair of parameter sets, respectively, and two groups of parameters G12 and G13 are formed S24 and S25.

Each formed groups of G12 and G13 parameters are processed in the unit for assessing person's functional state using a mathematical model to obtain S26 and S27 person's functional state assessment values E12 and E13. Said mathematical model is selected from a group consisting of a decision tree, a neural network, and one of three regressions: linear, logistic, and probit, to obtain the value Eij of the person's functional state assessment for each formed group Gij of parameters.

Each obtained value E12 and E13 is compared S28 with the tabular assessment values selected from the table for the corresponding therapeutic test. The table for the corresponding therapeutic test comprises a set of functional state assessment values from 1 to M and corresponding descriptions for each assessment value as well as standard recommendations. The compliance of the obtained functional state assessment value Eij to one of the assessment values provided in the table of the corresponding therapeutic test is identified.

Recommendations are formed S29 for the person based on the functional state descriptions and standard recommendations provided in the table for the identified functional state assessment values.

The implementation of the formed recommendations is monitored S30, and adjusted S32 based on the monitoring data.

The person's functional state is corrected S31 by implementation of the formed recommendations and adjustment S32 of it if necessary. The process of correction is described below.

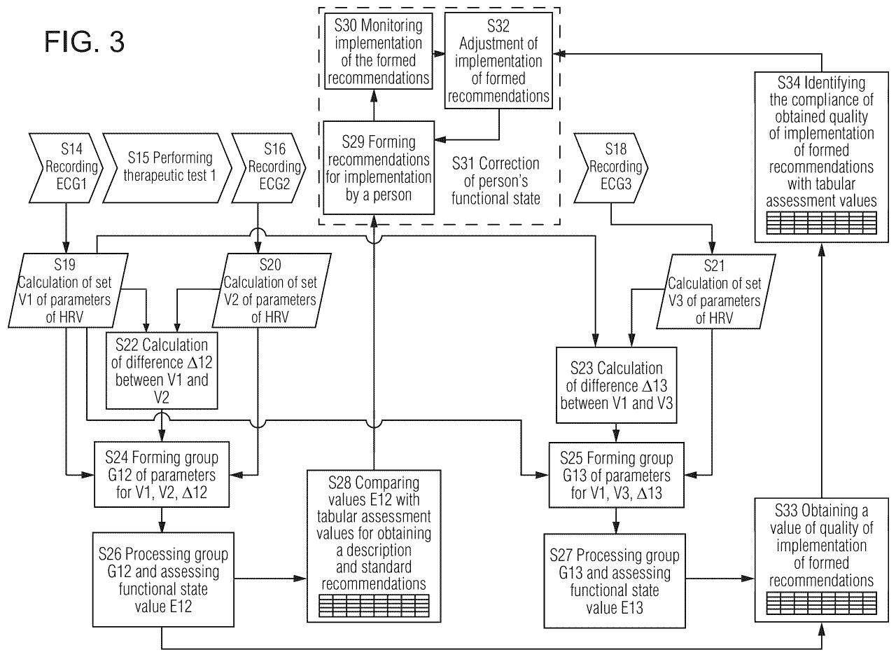

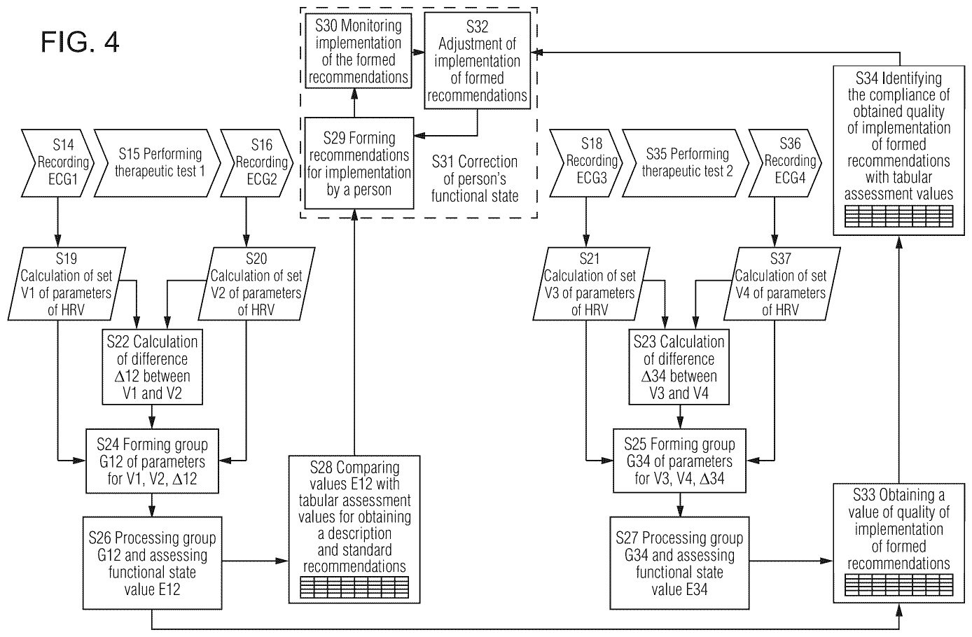

In a second embodiment of the claimed method it is possible to assess the quality of implementation of the formed recommendations (FIG. 3 and FIG. 4).

FIG. 3 shows an embodiment in which two ECGs are first recorded S14, S16, parameters V1 and V2 are calculated, the difference Δ12 is calculated, a group G12 of parameters is formed and processed S24, and the person's functional state assessment value E12 before the recommendations are implemented is obtained S26; E12 is compared S28 with the tabular assessment values. Recommendations are then formed S29 and implemented as described above. After that the third ECG3 is recorded S18, V3 is calculated, difference Δ13 is calculated, a group G13 of parameters is formed S25. Group G13 is processed and the person's functional state assessment value E13 is obtained S27 after following the recommendations.

The obtained person's functional state assessment value E12 before receiving the recommendations and the person's functional state assessment value E13 after implementing the formed recommendations are then compared S33 with the assessment values of the table to obtain the quality assessments value of implementation of recommendation. Said table of quality assessment values comprises assessment values before receiving the recommendations, assessment values after implementing the recommendations, and the corresponding quality assessment values.

Additionally, the correspondence of the obtained quality assessment value to the table descriptions of quality assessment values of implementation of recommendations is identified, wherein the table descriptions of quality assessment values of implementation of recommendation comprises a set of assessment values from 1 to Q and corresponding descriptions for each quality assessment value of of implementation of recommendation.

FIG. 4 shows an embodiment of the method that provides the ability to obtain a quality assessment value of implementation of recommendation in case where four ECGs are recorded.

Similar to the above-described embodiment, first two ECGs are recorded S14, S16, parameters V1 and V2 are calculated, the difference Δ12 is calculated, group G12 of parameters is formed and processed S24, and the person's functional state assessment E12 value before the recommendations are implemented is obtained S26, which is compared S28 with the tabular assessment values.

Then recommendations S29 are implemented, as described above. After the recommendations are implemented, a third ECG3 is recorded S18, and after the person performs S35 the therapeutic test, a fourth ECG4 is recorded S36, for which sets V3 and V4 of HRV parameters are also calculated S21 and S37, the difference Δ34 is calculated S23, group G34 of parameters is formed S25, which is processed, and the E34 value of the functional state assessment after the recommendations are implemented is obtained S27.

Then, the obtained person's functional state assessment value E12 before receiving the recommendations and the person's functional state assessment value E34 after implementing the formed recommendations are compared with the tabular values to obtain S33 the quality assessments values of implementation of the recommendations. Said table quality assessments values comprises the assessment value before receiving the recommendations, the assessment value after implementation of the recommendations and the corresponding quality assessment values of implementation of the recommendations.

Additionally, the compliance of the obtained quality assessment value of implementation of recommendations with the table descriptions is performed. The table of descriptions of quality assessment values of implementation of the recommendations comprises a set of assessment values from 1 to Q and corresponding descriptions for each assessment value (see examples).

The third embodiment of the method differs from the first and second embodiments in that a Fourier transform is performed for all N recorded ECGs to obtain a Fourier amplitude spectrum for each recorded ECG.

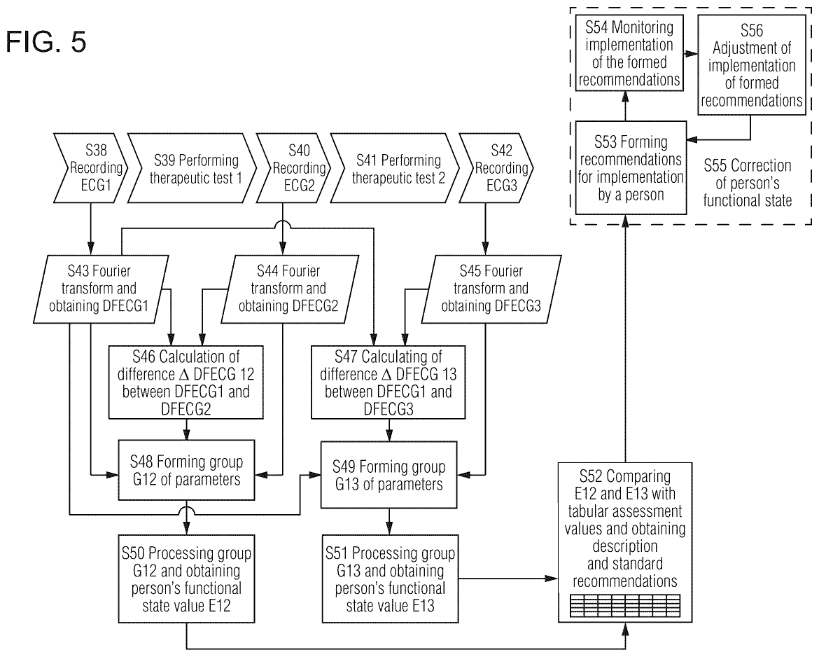

In this embodiment (FIG. 5), three electrocardiograms are recorded from at least the first lead, wherein the first ECG1 is initially recorded S38, the second ECG2 is recorded S40 after the person has completed S39 the first therapeutic test, and each subsequent ECG. In this case ECG3 is recorded S42 after the person has completed S41 the subsequent, in this case second, therapeutic test.

In the described embodiment, two pairs of recorded ECGs are selected, i.e. ECG1 and ECG2, and ECG1 and ECG3.

A Fourier transform is performed for each ECG of the selected pair, ECG1 and ECG2, to obtain a Fourier amplitude spectrum for each recorded ECG, and each obtained Fourier amplitude spectrum is discretized S43, S44, and S45 to obtain a discrete form of Fourier amplitude spectrum (DFECG) for ECG1, ECG2, and ECG3, respectively.

The difference ΔDFECG12 between the selected pair of discrete forms of Fourier amplitude spectrums, DFECG1 and DFECG2 is calculated S46, and the difference ΔDFECG13 between the selected pair of discrete forms of Fourier amplitude spectrums, DFECG1 and DFECG3 is calculated S47.

Then a group G12 of parameters is formed S48 for the pair ECG1 and ECG2, consisting of DFECG1 and DFECG2 and their calculated difference Δ12; and a group G13 of parameters is formed S49 for the selected pair ECG1 and ECG3, consisting of DFECG1 and DFECG3 and their calculated difference Δ13.

The formed group of G12 parameters is processed S50 in the functional state assessment unit, using a mathematical model selected from a group consisting of a decision tree, a neural network, and one of three regressions-linear, logistic, and probit to obtain an S50 value of E12 for the person's functional state assessment. The formed group of G13 parameters is also processed in the functional state assessment unit, using a mathematical model selected from a group consisting of a decision tree, a neural network, and one of three regressions-linear, logistic, and probit to obtain S51 the value of E13 for the person's functional state assessment.

Each obtained functional state assessment value of E12 and E13 of the corresponding selected pair of recorded ECG, is compared S52 with the tabular assessment values selected from the table for the corresponding therapeutic test. The table for the corresponding therapeutic test comprises a set of functional state assessment values from 1 to M and a corresponding description for each assessment value of the corresponding functional state, as well as standard recommendations. The correspondence of each obtained value of E12 and E13 for the functional state assessment to one of the assessment values provided in the table for the corresponding therapeutic test is identified.

Recommendations are formed S53 for the person, based on the functional state descriptions and standard recommendations provided in the table for the obtained functional state assessment values for all selected pairs of recorded ECG1, ECG2, and ECG3.

The implementation of the formed recommendations is monitored S54, and adjusted S56 based on the monitoring data.

As a result, and according to the claimed invention the person's functional state is corrected S55 by implementing the formed recommendations.

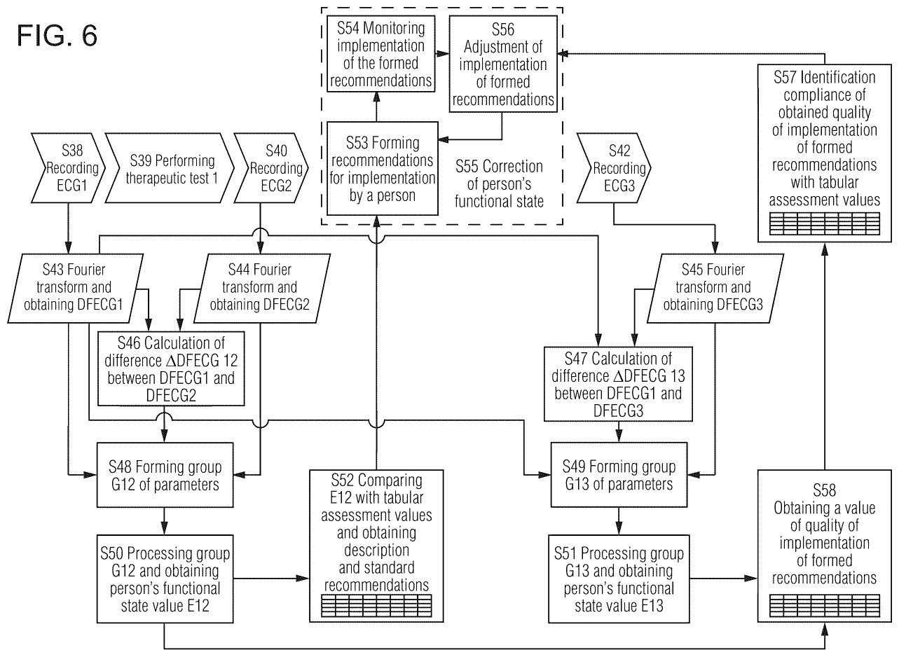

The third embodiment of the method also provides the ability to obtain a quality assessment value of implementation of the formed recommendations (FIG. 6).

FIG. 6 shows an embodiment where two ECG1 and ECG2, are recorded S38, S40, a Fourier transform is performed for each ECG1 and ECG2 to obtain a Fourier amplitude spectrum, and each obtained Fourier amplitude spectrum is discretized S43, S44 to obtain a discrete form of the Fourier amplitude spectrum (DFECG) for ECG1 and ECG2, respectively.

The difference ΔDFECG12 is then calculated and a group G12 of parameters is formed S48. Group G12 is processed S50 to obtain the person's functional state assessment value E12 before the recommendations are implemented by the person. Said value E12 is compared S52 with the tabular assessment values. Recommendations S53 are then formed and implemented according to the method as described above.

After the person implements the recommendations, a third ECG3 is recorded, a Fourier transform is performed on ECG3 to obtain a Fourier amplitude spectrum, and the resulting Fourier amplitude spectrum is discretized, resulting in a discrete form of Fourier amplitude spectrum (DFECG3) for ECG3. The difference ΔDFECG13 is then calculated, and a group of parameters, G13, is formed, which is processed S51 to obtain the functional state assessment value, E13. The obtained value characterizes the person's functional state assessment value after implementation of the recommendations.

The obtained person's functional state assessment value E12 before receiving the recommendations and the person's functional state assessment value E13 after implementing the formed recommendations are then compared with the tabular assessment values to obtain S58 a quality assessment value of implementation of the recommendations. Said table comprises the assessment value before receiving the recommendations, the assessment value after implementing the recommendations, and the corresponding quality assessment value of implementation of the formed recommendations.

Additionally, the obtained quality assessment value of implementation of the formed recommendations is compared with the table of descriptions of quality assessment values. The table of descriptions of quality assessment values comprises a set of quality assessment values from 1 to Q and corresponding descriptions for each quality assessment value of implementation of the recommendations.

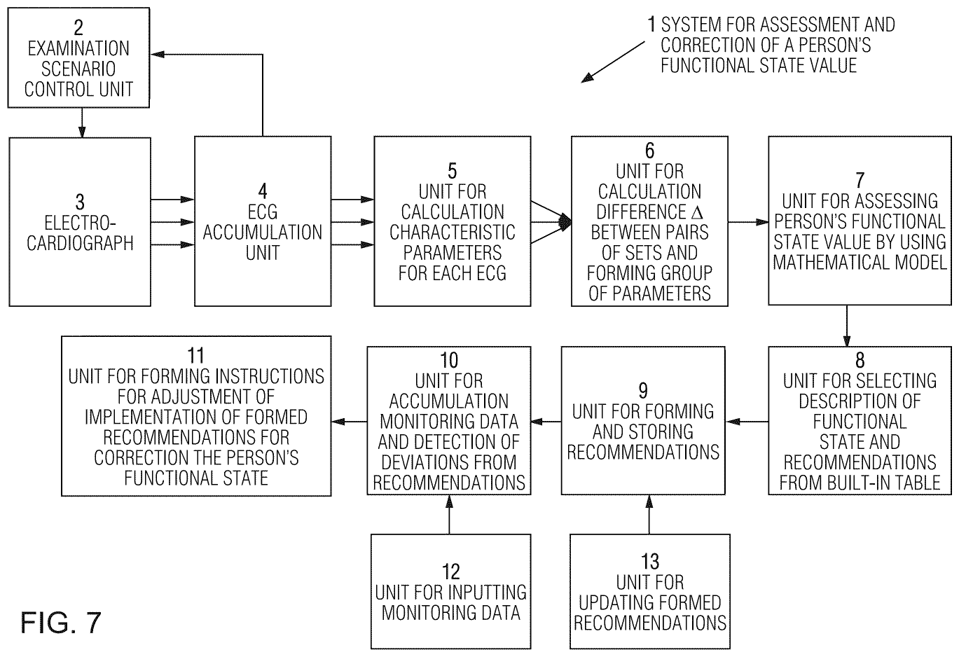

A system 1 (FIG. 7) for assessment and correction a functional state of a person is disclosed to implement the claimed method. The system comprises examination scenario control unit 2, connected to electrocardiograph configured to record a set of electrocardiograms (ECG) of at least the first lead before and after the person has performed at least one therapeutic test. The system comprises ECG accumulation unit 4 connected in turn to electrocardiograph 3.

The system 1 further comprises connected in series:
unit 5 for calculating sets of characteristic parameters for each of the recorded ECGs, connected to ECG accumulation unit 4; unit 6 for calculation difference Δ between pairs of sets of characteristic parameters and forming group G of parameters; unit 7 for assessing person's functional state value, configured to process the formed group of parameters and obtain person's functional state assessment value by using a mathematical model selected from a group consisting of a decision tree, a neural network, one of three regressions-linear, logistic and probit, unit 7 is connected to unit 6.

The system 1 also comprises unit 8 for selecting a description of functional state and recommendations from a built-in table of corresponding therapeutic test and forming standard recommendations based on the resulting assessment value. The table comprises a set of person's functional state assessing values from 1 to M, along with descriptions and standard recommendations for each assessment value. Unit 9 for forming and storing recommendations is connected to unit 8.

The system comprises unit 10 for accumulation of monitoring data, configured to accumulate the monitoring data of implementation of the formed recommendations and further to detect deviations from the recommendations.

The system 1 also comprises unit 11 for forming instructions for adjustment of implementation of formed recommendations for correction a person's functional state.

The system 1 comprises unit 12 for inputting monitoring data, connected to unit 10.

The system further comprises unit 13 for updating formed recommendations, connected to unit 9 for forming and storing recommendations. The updating information may be formed by a physician or a computer system.

Unit 5 for calculating sets of characteristic parameters is configured to calculate a set of heart rate variability parameters. In this embodiment, the system utilizes a cardiograph 3 capable of determining the timing of heart contractions.

An alternative embodiment is possible wherein unit 5 for calculating sets of characteristic parameters is configured to determine the timing of heart contractions.

An alternative embodiment is possible wherein unit 5 for calculating sets of characteristic parameters is configured to calculate a discretized form of ECG (DFECG).

The method for assessment and correction of a person's functional state, according to the first embodiment of the invention, is disclosed in Examples 1-5 below.

### Example 1

Example 1 is concerned a method for assessment and correction of an athlete's functional state, implemented as follows.

The athlete's first ECG was recorded at 10:00 a.m. in the medical clinic after a night's rest, after that the athlete performed a therapeutic test, i.e. a prescribed physical exercise 30 squats in 45 seconds, and a second ECG was immediately recorded.

The ECG1 and ECG2 were processed using a cardiograph. A set of V1 and V2 heart rate variability (HRV) parameters were calculated for each of the two recorded ECG1 and ECG2; the difference Δ between the corresponding parameters of the obtained sets of V1 and V2 heart rate variability were calculated. Group G of parameters consisting of a set of V1 HRV parameters, a set of V2 HRV parameters, and the calculated difference Δ between the sets of parameters was formed.

The resulting group of G parameters was then processed in unit for assessing person's functional state value by using mathematical model described above, to obtain a functional state assessment value for the athlete. The result “3” was obtained.

The resulting functional state assessment value was compared with the tabular assessment values selected from Table 1 for the corresponding therapeutic test, that is a physical exercise consisting of 30 squats in 45 seconds.

Table 1 (shown below) comprises a set of functional state assessment values from 1 to M=6, along with text descriptions for each assessment value for the corresponding functional state, as well as standard recommendations.

Table 1 comprises descriptions of six states selected from a group consisting of critical overstrain, overexertion, fatigue, incomplete recovery, complete recovery, and supercompensation.

In the example under consideration, an assessment value of 3, according to Table 1, corresponds to the person's functional state of “fatigue”, i.e., a state that develops as a result of physical or psychological overstrain, causing functional disorders of the nervous system.

TABLE 1

| Assessment | | |
|---|---|---|
| Value | State and Description | Standard Recommendations |
| 6 | Supercompensation | Perform exercises in the anaerobic |
| | A state of recovery with an increase | zone (HR 110-120% of the ATHR), |
| | in the initial level of functional | but aerobic, restorative, and |
| | capacity, characterized by an | rehabilitative exercises are also |
| | increase in the efficiency of exercise | permitted |
| | performed over a certain period of | |
| | time | |
| 5 | Full Recovery | Perform exercises in the aerobic and |
| | A state of recovery with an increase | developmental zones (HR 95-110% |
| | in the initial level of functional | of the ATHR), but restorative and |
| | capacity, characterized by an | rehabilitative exercises are also |
| | increase in the efficiency of work | permitted |
| | performed over a certain period of | |
| | time | |
| 4 | Incomplete Recovery | Perform exercises in the aerobic zone |
| | A state resulting from activity and | with a small percentage of time spent |
| | characterized by a temporary | in the developmental zone (HR 90- |
| | decrease in the body's functional | 100% of the ATHR), but restorative |
| | capacity, characterized by a decrease | and rehabilitative exercise is also |
| | in the effectiveness of the exercise | permitted |
| | performed over a certain period of | |
| | time | |
| 3 | Fatigue | Perform exercises exclusively in the |
| | A state being a result of physical or | aerobic zone (HR 80-95% of the |
| | psychological overstrain, | ATHR), but restorative and |
| | determining functional impairment | rehabilitative exercise is also |
| | of the nervous system | permitted |
| 2 | Overstrain | Perform exercises in the recovery or |
| | A state of profound physical, mental, | aerobic zones (HR 70-80% of the |
| | and emotional exhaustion resulting | ATHR), but rehabilitative exercise is |
| | from prolonged or intense stress. | permitted |
| | This state is a result of prolonged | |
| | physical or mental overstrain | |
| 1 | Significant (critical) overstrain | Complete prohibition of physical |
| | Pathological exhaustion of the body's | activity |
| | regulatory systems, specialist | |
| | intervention and/or consultation is | |
| | required | |

The anaerobic threshold heart rate (ATHR) in Table 1 is the heart rate at the anaerobic threshold. Heart rate is a physical quantity obtained by measuring the number of cardiac contractions per unit of time and is measured in beats per minute (bpm). The anaerobic threshold (ATHR) is an indicator of the efficiency of bioenergetic mechanism, characterizing the onset of oxidative uncompensated activation of the anaerobic energy production process during muscular activity.

It was determined whether the athlete's functional state value of “3” corresponded to one of the values from 1 to M=6, as shown in Table 1.

Recommendations for the athlete were based on the identified correspondence between the functional state value of “3” and the standard recommendation: “perform exercises exclusively in the aerobic zone (HR 80-95% of the ATHR), but restorative and rehabilitative exercise is also permitted”. In the example, cycling and running were recommended for at least 30 minutes, or walking recovery and rehabilitation exercises for at least 30 minutes. The athlete chose to exercise on a stationary bike.

Heart rate was measured during the exercise to monitor implementation of the recommendations.

Functional state was controlled through adjusted implementation of the recommendations, i.e., increasing or decreasing pedaling cadence during cycling. Heart rate was measured during the exercise using a Polar H10 heart rate monitor.

During the exercise on the stationary bike, the heart rate exceeded the recommended value of 80-95% of the ATHR, so the pedaling cadence was lowered during the exercise.

As a result, an improvement in the functional state of the nervous system was determined by the sports physician, expressed in an improvement in the general emotional state (mood) and a decrease in the level of tension in the body-physical overstrain.

### Example 2

Example 2 is concerned a method of assessment and correction of the functional state of a physical training participant (an individual actively involved in physical training, but not an athlete), implemented as follows.

The first ECG was recorded after morning exercises in a sports medicine doctor's office. The participant then performed a therapeutic test, particularly, a respiratory load, i.e., 30 forced inhalations and slow exhalations over 45 seconds. A second ECG was then recorded.

The resulting ECGs 1 and 2 were processed. For each of the two ECGs, the corresponding sets of V1 and V2 heart rate variability (HRV) parameters were calculated. The difference Δ between the corresponding parameters of the obtained V1 and V2 heart rate variability sets was calculated.

Group G was formed, group G consists of a V1 set of HRV parameters, a V2 set of HRV parameters, and calculated difference Δ between the parameter sets. The resulting group G was then processed in unit for assessing person's functional state value by using mathematical model to obtain a functional state assessment value. The result “5” was obtained.

The obtained functional state assessment value was compared with the table 2 of assessment values. The table comprises a set of functional state assessment values from 1 to 6, along with corresponding text descriptions for each assessment value for the corresponding functional state, as well as standard recommendations.

The obtained functional state assessment value was compared with one of the assessment values provided in Table 2, corresponding to six states selected from a group consisting of a pathological state, gas exchange system overstrain, insufficient gas exchange compensation, moderate gas exchange compensation, good gas exchange compensation, and supercompensation. In this example, an assessment value of 5 corresponds to the athlete's functional state: “good gas exchange compensation”.

TABLE 2

| Assessment | | |
|---|---|---|
| Value | State and Description | Standard Recommendations |
| 6 | Supercompensation | Perform physical activity: |
| | The functional capacity of the lungs | maximum-intensity anaerobic, |
| | allows for enhanced gas exchange by | aerobic-anaerobic at the ATHR, |
| | fully performing an exhalation before | and low- and moderate-intensity |
| | the next inhalation. No deterioration of | aerobic. |
| | lung volumes: tidal volume, | |
| | inspiratory reserve volume, expiratory | |
| | reserve volume, and residual volume. | |
| | No deterioration of lung capacity: vital | |
| | capacity, inspiratory capacity, | |
| | functional residual capacity, and total | |
| | lung capacity | |
| 5 | Good Gas Exchange Compensation | Perform physical activity: |
| | No deterioration of the structures that | aerobic-anaerobic at the ATHR, |
| | ensure lung function. No deterioration | low- and moderate-intensity |
| | of external respiration. Virtually no | aerobic. |
| | obstruction of the airway | |
| 4 | Moderate gas exchange | Perform low- and moderate- |
| | compensation | intensity aerobic physical activity |
| | Moderate deterioration of the | |
| | structures that ensure lung function. | |
| | Moderate deterioration of external | |
| | respiration. Moderate obstruction of | |
| | airway patency. | |
| 3 | Insufficient gas exchange | Perform low-intensity aerobic |
| | compensation | physical activity |
| | The functional capacity of the lungs | |
| | does not allow for enhanced gas | |
| | exchange due to incomplete exhalation | |
| | before the next inhalation. Moderate | |
| | deterioration of lung volumes: tidal | |
| | volume, inspiratory reserve volume, | |
| | expiratory reserve volume, residual | |
| | volume. Moderate deterioration of | |
| | lung capacity: vital capacity, | |
| | inspiratory capacity, functional | |
| | residual capacity, total lung capacity | |
| 2 | Overstraining of gas exchange | Perform exercise in a state of |
| | systems | relative rest; consult a specialist to |
| | The presence of possible abnormalities | rule out pathological processes |
| | in the structures that form the chest | |
| | (bones, muscles, nerves) that reduce | |
| | lung function. The presence of | |
| | potentially significant airway | |
| | obstruction. Abnormalities in lung | |
| | volumes: tidal volume, inspiratory | |
| | reserve volume, expiratory reserve | |
| | volume, residual volume. | |
| | Abnormalities in lung capacity: vital | |
| | capacity, inspiratory capacity, | |
| | functional residual capacity, total lung | |
| | capacity | |
| 1 | Pathological reaction | Consult a specialist to rule out |
| | Abnormalities in the structures that | pathological processes and permit |
| | form the chest (bones, muscles, | physical activity |
| | nerves). Pathological obstruction of | |
| | the airway. Significant deterioration of | |
| | lung capacity: tidal volume, | |
| | inspiratory reserve volume, expiratory | |
| | reserve volume, residual volume. | |
| | Significant deterioration of lung | |
| | capacity: vital capacity, inspiratory | |
| | capacity, functional residual capacity, | |
| | total lung capacity | |

Recommendations were formed for the physical training participant, based on the identified correspondence of the obtained value equal to 5 and the tabular assessment value, as well as the descriptions provided in Table 2. The recommendations were based on a standard recommendation: “Perform physical activity: aerobic-anaerobic at the ATHR, low- and moderate-intensity aerobic”.

In this example, the recommended strength exercises were: lifting a 100 kg weight and high-speed cycling (speeds above 30 km/h, as determined by the speedometer).

A blood saturation and heart rate were measured during the recommendations to monitor a compliance with the recommendations.

Correction of the person's functional state was performed by adjusted implementation of recommendations, that is, increasing or decreasing the number of repetitions of the weightlifting exercise and decreasing pedaling speed during the cycling exercise. Blood saturation and heart rate were measured during the 100 kg weightlifting exercise and high-speed cycling. Monitoring showed that saturation dropped from the recommended range to below 95% during the weightlifting session, so the physician reduced the repetition count to 1 repetition.

Monitoring showed that a blood pressure changed beyond the recommended range, with systolic pressure exceeding 180 mmHg and diastolic pressure falling below 50 mmHg during high-speed cycling. Therefore, the trainer set the pedaling speed to less than 15 km/h.

As a result, lung function was maintained.

### Example 3

Example 3 is concerned a method for assessment and correction of the functional state of a clinical patient undergoing treatment with a general practitioner for an acute respiratory viral infection (ARVI). The method was implemented as follows.

The first ECG was recorded in a hospital after the minimum diagnostic therapeutic procedure, including profiling and examination. The therapeutic test was performed, in particular, an intravenous pharmacological procedure, namely, the intravenous administration of 200 ml of 0.9% sodium chloride solution. Then the second ECG was recorded. The influence was performed to reduce intoxication and improve complete blood count results.

The obtained ECGs 1 and 2 were processed. For each of the two ECGs, corresponding sets of V1 and V2 heart rate variability (HRV) parameters were calculated. A difference Δ between the corresponding parameters of the obtained V1 and V2 heart rate variability sets was calculated. Group G was formed consisting of the V1 set of HRV parameters, the V2 set of HRV parameters, and the calculated difference Δ.

The resulting group of parameters was then processed in the functional state assessment unit to obtain a functional state assessment value. The functional state assessment value “4” was obtained.

The obtained functional state assessment value was compared with table 3 of assessment values. Table 3 (below) comprises a set of patient's functional state assessment values from 1 to M=5, along with corresponding descriptions for each assessment value for the corresponding functional state and standard recommendations. The resulting functional state assessment value was compared with one of the assessment values provided in Table 3.

In this example, an assessment value of 4 corresponds to the patient's functional state: “favorable improvement”, an increase in the initial level of functional capacity. The functional state description and corresponding recommendations are provided in Table 3.

TABLE 3

| Assessment | | |
|---|---|---|
| Value | State and Description | Standard Recommendations |
| 5 | Unfavorable Improvement | Stop further influences. Observation is |
| | Excessive increase in baseline | required to rule out the development of |
| | functional capacity | adverse effects on the person body's |
| | | functional capacity |
| 4 | Favorable Improvement | Stop the influence. The influence |
| | Increase in baseline functional | performed determines further actions |
| | capacity. Discontinue further | taking into account the favorable change in |
| | influences | the person body's functional state. |
| 3 | Unchanged State No | A decision needs to be made about |
| | significant change in baseline | additional influences taking into account |
| | functional capacity | the absence of a change in the person |
| | | body's functional state |
| 2 | Favorable Deterioration | Further influences may be discontinued if a |
| | A decrease in the initial level | favorable change in the person body's |
| | of functional capacity without | functional capacity is achieved despite the |
| | a reduction in the effectiveness | deterioration in the individual's functional |
| | of exercises performed over a | state, or continued if an improvement in |
| | certain period of time | the person body's functional state is |
| | | required |
| 1 | Unfavorable Deterioration | Stop all influences. Immediate clinical |
| | An excessive decrease in the | assessment of the individual's health and, if |
| | initial level of functional | necessary, contact emergency medical |
| | capacity | services to determine the diagnosis of the |
| | | person body to rule out pathological states |

Recommendations were formed based on the identified correspondence between the obtained functional state assessment 4 and the tabular assessment values and the text descriptions of the functional state values provided in Table 3. These recommendations were based on the standard recommendation: “Stop the influence. The influence performed determines further actions taking into account the favorable change in the person body's functional state”. Palpation, percussion, and auscultation were used to monitor compliance with the recommendations.

Functional state was corrected through adjusted implementation of recommendations, i.e., discontinuing the intravenous procedure.

After completion of the intravenous procedure, during rest, no deviations from practical norms were detected based on palpation, percussion, and auscultation.

As a result, the general practitioner determined an improvement in the body's functional state, reflected in significantly improved state of the patient, a reduction in cold symptoms, and improved clinical blood test results.

### Example 4

Example 4 is concerned a method of assessment and correction of the functional state of a patient undergoing rehabilitation, implemented as follows.

The first ECG was recorded, followed by a therapeutic test. The therapeutic test consisted of 30 squats for 45 seconds, followed by physical therapy. A magnetic therapy session was used as the therapeutic test, and then a second ECG was recorded. The obtained ECG1 and ECG2 were processed. For each of the ECG, the corresponding sets of V1 and V2 heart rate variability (HRV) parameters were calculated. The difference Δ between the corresponding parameters of the obtained V1 and V2 heart rate variability sets was calculated. Group G was formed consisting of the V1 set of HRV parameters, the V2 set of HRV parameters, and the calculated difference Δ between the parameter sets.

The obtained group of parameters was then processed in the functional state assessment unit to obtain a functional state assessment value. The obtained assessment value was “2”.

The resulting functional state assessment value was compared with the table of assessment values. Table 4 (shown below) comprises a set of functional state assessment values from 1 to M=4 and corresponding text descriptions for each assessment value for the corresponding functional state, as well as standard recommendations.

The obtained functional state assessment value was determined to correspond to one of the assessment values provided in Table 4. An assessment value “2” corresponded to the patient's functional state: “fatigue”, a state developing as a result of physical or psychological overstrain, determining functional disorders of the nervous system.

TABLE 4

| Assessment | | |
|---|---|---|
| Value | State and Description | Standard Recommendations |
| 4 | Supercompensation | Perform exercises in the anaerobic |
| | A state of recovery with an increase in | zone (HR 110-120% of the |
| | the initial level of functional capacity, | ATHR), but aerobic, restorative, |
| | characterized by an increase in the | and rehabilitative loads are also |
| | efficiency of exercise performed over a | permitted |
| | certain period of time | |
| 3 | Recovery | Perform exercises in the aerobic |
| | A state of recovery to the initial level | (HR 95-110% of the ATHR) and |
| | of the body's functional capacity, | developmental zones (HR 90-100% |
| | characterized by an increase in the | of the restorative and rehabilitative |
| | efficiency of exercise performed over a | exercises are also permitted |
| | certain period of time, as well as a state | |
| | being a result of activity and a | |
| | temporary decrease in the level of the | |
| | body's functional capacity, | |
| | characterized by a decrease in the | |
| | efficiency of exercise performed over a | |
| | certain period of time | |
| 2 | Fatigue | Perform exercises exclusively in |
| | A state that develops as a result of | the aerobic zone (heart rate 80-95% |
| | physical or psychological overstrain, | of the ATHR), but restorative and |
| | causing functional impairment of the | rehabilitative exercise is also |
| | nervous system | permitted |
| 1 | Overstrain | Perform exercises in the restoration |
| | A state of profound physical, mental, | or aerobic zones (heart rate 70-80% |
| | and emotional exhaustion resulting | of the ATHR), but rehabilitative |
| | from prolonged or intense stress, which | exercise is permitted. A complete |
| | develops in a person as a result of | ban on physical activity is possible |
| | prolonged physical or mental | after consultation a specialist |
| | overstrain, as well as pathological | |
| | exhaustion of the body's regulatory | |
| | systems, requiring influence and/or | |
| | consult of a specialist | |

Recommendations were formed based on the identified correspondence between the obtained functional state value “2” and the tabular assessment value, as well as the text descriptions of functional state provided in Table 4. These recommendations were based on a standard recommendation: “Perform exercises exclusively in the aerobic zone (heart rate 80-95% of the ATHR), but restorative and rehabilitative exercise is also permitted”.

In this example, the patient was recommended to perform exercises on an elliptical and a rowing machine for 30 minutes in a single, continuous session.

The physician performed palpation and percussion during the implementation of the recommendations to monitor compliance with the recommendations.

Functional state was corrected through adjusted implementation of the recommendations, that is, by increasing or decreasing the number of steps on the elliptical or the number of strokes on the rowing machine during the 30-minute session. During exercises on the elliptical and rowing machines, palpation and percussion revealed heart rate increases above the recommended 140 beats per minute. Therefore, the physician recommended reducing the number of steps on the elliptical and the number of strokes on the rowing machine.

As a result, the physician observed a reduction in the patient's physical and psychological stress, improved functional capabilities, mood, and subjective perceptions of health.

### Example 5

Example 5 is concerned a method of assessment and correction of the functional state of an athlete, implemented as follows.

The first ECG was recorded, followed by a therapeutic test. The therapeutic test consisted of 30 push-ups in 45 seconds, and then a second ECG was recorded. The obtained ECG1 and ECG2 were processed. The corresponding sets of V1 and V2 heart rate variability (HRV) parameters were calculated for each of the two ECGs. The difference Δ between the corresponding parameters of the obtained V1 and V2 heart rate variability sets was calculated. A parameter group was formed consisting of the V1 set of HRV parameters, the V2 set of HRV parameters, and the calculated Δ difference between the parameter sets.

The resulting parameter group was then processed in the functional state assessment unit to obtain a functional state assessment value. The obtained functional state assessment value was “7.4”.

The resulting functional state assessment value was compared with the tabular assessment values selected from Table 5 for the corresponding therapeutic test of 30 push-ups in 45 seconds. Table 5 (shown below) comprises a set of functional state assessment values from 0 to M=10, along with corresponding text descriptions for each assessment value and standard recommendations.

In the example under consideration, a 7.4 assessment value corresponded to the athlete's “good functional state”. This refers to a state of recovery to the body's initial level of functional capacity, characterized by an increase in the effectiveness of work performed over a certain period of time, as well as a state resulting from activity and a temporary decrease in the body's functional capacity, characterized by a decrease in the effectiveness of work performed over a certain period of time.

TABLE 5

| Assessment | | |
|---|---|---|
| Value | State and Description | Standard Recommendations |
| 7.5 to 10 | Best Functional State | Perform exercises in the anaerobic |
| | Recovery with an increase in the initial | zone (HR 110-120% of the |
| | level of functional capabilities, | ATHR), but aerobic, restorative, |
| | characterized by an increase in the | and rehabilitative exercises are |
| | efficiency of exercises performed over | also allowed |
| | a certain period of time | |
| 5 to 7.49 | Good Functional State | Perform exercises in the |
| | Recovery to the initial level of the | developmental (HR 95-110% of |
| | body's functional capabilities, | the ATHR) and aerobic (HR 90- |
| | characterized by an increase in the | 100% of the ATHR)zones, but |
| | efficiency of work performed over a | restorative and rehabilitative |
| | certain period of time, as well as a | exercises is also permitted |
| | state arising as a result of activity and | |
| | by a temporary decrease in the level of | |
| | the body's functional capabilities, | |
| | characterized by a decrease in the | |
| | efficiency of exercise performed over | |
| | a certain period of time | |
| 2.5 to 4.99 | Satisfactory functional state | Perform exercises exclusively in |
| | A state developing as a result of | the aerobic zone (heart rate 80- |
| | physical or psychological overstrain, | 95% of ATHR), but restorative |
| | causing functional impairment of the | and rehabilitative exercise is also |
| | nervous system | permitted |
| 0.01 to 2.49 | Worst functional state | Perform exercises in the |
| | A state of profound physical, mental, | restorative or aerobic zones (heart |
| | and emotional exhaustion resulting | rate 70-80% of ATHR), but |
| | from prolonged or intense stress that | rehabilitative exercise is |
| | develops in a person as a result of | permitted. A complete ban on |
| | prolonged physical or mental | physical activity is possible after |
| | overstrain, as well as pathological | consultation with a specialist |
| | exhaustion of the body's regulatory | |
| | systems, requiring specialist influence | |
| | and/or consultation | |

Recommendations for the athlete were formed based on the identified correspondence between the obtained functional state value of 7.4 and the tabulated assessment value, as well as the text descriptions of functional state provided in the table. These recommendations were based on a standard recommendation: “Perform exercises in the developmental (HR 95-110% of the ATHR) and aerobic (HR 90-100% of the ATHR) zones, but restorative and rehabilitative exercises is also permitted”.

In this example, performing a 93 kg deadlift was recommended.

To monitor compliance with the recommendations, body temperature was measured and the level of blood lactate accumulation was simultaneously determined during the implementation of the recommendations.

Functional state was corrected through adjusted implementation of the recommendations, that is, by increasing the number of sets to ensure work in the developmental heart rate zone, with one set comprises 6 deadlift repetitions.

The athlete's body temperature was measured and found to be 36.8° C. during the deadlift, and the blood lactate level did not exceed 5 mmol/L. Therefore, the sports trainer recommended performing four sets.

The method of assessment and correction of a person's functional state, according to the second embodiment of the invention, is illustrated below by Examples 6-10.

### Example 6

Example 6 is concerned a method of assessment and correction of the functional state of a teenager actively involved in physical education.

The first ECG was recorded at rest in the school physician's office after school. The teenager then performed the first therapeutic test. The therapeutic test consisted of a prescribed physical load of 30 arm swings in 45 seconds, and then a second ECG was recorded. The teenager then performed a second therapeutic test. This therapeutic test consisted of 20 forced inhalations and slow exhalations for 45 seconds, followed by a third ECG.

The obtained ECGs 1, ECG2, ECG3 were processed, for which the corresponding sets of V1, V2, V3 heart rate variability (HRV) parameters were calculated for each of the three recorded ECGs, the difference Δ12 and Δ13 between the corresponding parameters of the obtained sets V1 and V2, V1 and V3 heart rate variability was calculated.

Two groups of parameters were formed, one group consisting of the set V1 of HRV parameters, the set V2 of HRV parameters and the calculated difference Δ12 between the sets of parameters and a group of parameters consisting of the set V1 of HRV parameters, the set V3 of HRV parameters and the calculated difference Δ13.

The resulting parameter groups were processed in the functional state assessment unit to obtain a functional state assessment value. This yielded assessment value “5” for the first pair of HRV parameter sets, and value “3” for the second pair of sets.

The resulting assessment values were compared with the tabular assessment values selected from Table 1 for the first therapeutic test, i.e., 30 arm swings in 45 seconds, and Table 2 for the second therapeutic test, i.e., 20 forced inhalations and slow exhalations, along with the corresponding text descriptions for each assessment value of the corresponding functional state. The obtained functional state assessment values were compared to one of the assessment values provided in the tables for the corresponding therapeutic tests.

In this example, assessment values “5” and “3” correspond to the teenager's functional state: “full recovery” and “insufficient compensation of gas exchange”. The functional state description and corresponding standard recommendations are provided in Table 1 and Table 2.

Recommendations for individuals were based on the identified correspondence between the obtained functional state values 5 and 3 and the tabular assessment value, as well as the text descriptions of the functional state presented in Tables 1 and 2. These recommendations were based on the standard recommendation: “Perform exercises in the aerobic and developmental zones (HR 95-110% of the ATHR), but restorative and rehabilitative exercises are also permitted”, as is “Perform low-intensity aerobic physical activity”.

In the example described, a general recommendation was given for long walks in the fresh air for more than 30 minutes per continuous bout.

To monitor implementation, auscultation and blood pressure measurements were performed during the walk.

The patient's functional state was corrected by adjusted implementation of the recommendations, that is, by increasing or decreasing the walking speed. This was accomplished by auscultation and blood pressure measurements during the walk.

During the walk, auscultation revealed a heart rate below the recommended 100 beats per minute, so the trainer recommended increasing the walking speed.

### Example 7

Example 7 is concerned a method of assessment and correction of the functional state of a patient with chronic renal failure undergoing treatment by a physician.

The first ECG was recorded in a hospital, then a therapeutic test was performed. An intravenous pharmacological procedure was performed as the therapeutic influence, i.e. the administration of 200 ml of 0.9% sodium chloride solution with the addition of heparin to achieve anti-inflammatory and analgesic effects, after which a second ECG was recorded.

The recorded ECG1 and ECG2 were processed. The corresponding sets of V1 and V2 heart rate variability (HRV) parameters were calculated for each of the two ECGs. The difference Δ12 between the corresponding parameters of the obtained V1 and V2 heart rate variability sets was calculated. A group G was formed consisting of the V1 set of HRV parameters, the V2 set of HRV parameters, and the calculated Δ12 difference between the parameter sets.

The resulting parameter group was then processed in the functional state assessment unit to obtain the patient functional state assessment value. As a result, a functional state assessment value “4” was obtained.

The obtained functional state assessment value was compared with the table of assessment values. Table 6 (below) comprises a set of functional state assessment values from 1 to M=5, along with corresponding text descriptions for each assessment value for the corresponding functional state and standard recommendations. The resulting functional state assessment value was compared with one of the assessment values provided in Table 6.

In this example, a functional state assessment value of 4 corresponds to the patient's functional state: “favorable improvement”. Increase in initial functional capacity. The functional state description and corresponding recommendations are provided in Table 6.

TABLE 6

| Assessment | | |
|---|---|---|
| Value | State and Description | Standard Recommendations |
| 5 | Unfavorable Improvement | Conduct a functional assessment using |
| | Excessive increase in | physical activity. If the values equal to 1, 2, 3, |
| | baseline functional capacity | or 4 are obtained, a specialist consultation is |
| | | required to determine the validity of the |
| | | therapeutic influence. After this, additional |
| | | exercises may be performed to correct the |
| | | functional state. |
| 4 | Favorable Improvement | Conduct a functional assessment using |
| | Increase in baseline | prescribed physical activity. If the values |
| | functional capacity | equal to 1, 2, 3 are obtained, a specialist |
| | | consultation is required to determine the |
| | | validity of the therapeutic influence. After |
| | | this, additional influences may be performed |
| | | to correct the functional state |
| 3 | Unchanged State | Conduct a functional assessment using |
| | No significant changes in | prescribed physical activity. If the values |
| | baseline functional capacity | equal to 1 or 2 are obtained, a specialist |
| | | consultation is required to determine the |
| | | validity of the therapeutic influence. After |
| | | this, additional exercises may be performed to |
| | | correct the functional state |
| 2 | Favorable Deterioration | Conduct a functional assessment using a |
| | Decrease in baseline | prescribed physical activity. If the values |
| | functional capacity without | equal to 1 and 2 are obtained, consult a |
| | a decrease in the | specialist regarding the validity of the |
| | effectiveness of exercises | therapeutic influence, after which additional |
| | performed over a specified | exercises may be performed to correct the |
| | period of time | functional state |
| 1 | Unfavorable Deterioration | Urgent consultation with a specialist who will |
| | Excessive decrease in | take measures to manage the state of the |
| | baseline functional capacity | person who has performed the therapeutic test |

Recommendations were formed based on the identified correspondence between the obtained functional state value “4” and the tabular assessment values and the text descriptions of the functional state provided in Table 6. These recommendations were based on the standard recommendation: “Conduct a functional assessment using prescribed physical activity. If the values equal to 1, 2, 3 are obtained, a specialist consultation is required to determine the validity of the therapeutic influence. After this, additional influences may be performed to correct the functional state”.

After receiving the recommendations, i.e. after conducting the functional state assessment, ECG3 was recorded, and then a therapeutic test was performed by the patient-a physical activity of 10 squats in 45 seconds, and then ECG4 was recorded.

The obtained ECG3 and ECG4 were processed, the corresponding sets of V3 and V4 heart rate variability (HRV) parameters were calculated for each of the two recorded ECGs, the difference Δ34 between the corresponding parameters of the obtained sets of V3 and V4 heart rate variability was calculated, a group G of parameters was formed consisting of the set of V3 HRV parameters, the set of V4 HRV parameters and the calculated difference Δ34 between the sets of parameters.

The resulting group G of parameters was then processed in the functional state assessment unit to obtain a functional state assessment value.

An assessment value “3” was obtained, and then additional influence in the form of intravenous administration of 200 ml of Reamberin solution was recommended by the physician.

ECG was recorded during the intravenous administration of Reamberin solution to monitor implementation of recommendations.

Functional state was corrected through adjusted implementation of recommendations, i.e., continued intravenous infusion of Reamberin solution, for which an electrocardiogram was recorded during the administration.

An improvement of the functional state was determined on the recorded ECG during the intravenous infusion of Reamberin solution, based on the calculated HRV parameters. This result allowed the physician continuing an administration of the solution. As a result, inflammation was reduced and a persistent analgesic effect was achieved.

According to the second embodiment of the invention, it is possible to evaluate the quality of implementation of the recommendations. For this at least two ECGs are recorded, HRV parameters are calculated, the difference Δ between the HRV parameters is calculated, a group G of parameters is formed and processed, and the value E of the assessment of the functional state of the person before the implementation of the recommendations is obtained.

Recommendations were formed and implemented by realizing the method according to claim 14, then at least one additional ECG is recorded. Between each successive pair of ECGs, the person performs a therapeutic test, processing of the additionally recorded ECGs was carried out, and the functional state assessment value after the implementation of the recommendations was obtained.

The obtained person's functional state assessment value before receiving the recommendations and the person's functional state assessment value after implementation of the formed recommendations are compared with tabular assessment values to obtain quality assessment value of implementation of recommendations. The table of quality assessment values (Table 7) comprises assessment value before performing the recommendations, the assessment value after implementing the recommendations, and the corresponding quality assessment value of implementation of recommendations.

TABLE 7

| | Assessment of a person's functional state | | | | | |
|---|---|---|---|---|---|---|
| | before implementing recommendations | | | | | |
| Assessment of person's functional | 6 | 5 | 4 | 3 | 2 | 1 |
| state value after implementing | Assessments of quality of implementation | | | | | |
| recommendations | of recommendations values | | | | | |
| 6 | 3 | 3 | ? | ? | ? | ? |
| 5 | 3 | 3 | 3 | 5 | ? | ? |
| 4 | 4 | 4 | 3 | 4 | 5 | ? |
| 3 | 5 | 5 | 5 | 3 | 4 | 5 |
| 2 | 2 | 2 | 2 | 2 | 3 | 4 |
| 1 | 1 | 1 | 1 | 1 | 1 | 1 |

Further, the compliance of the obtained quality assessment value of implementation of recommendations is identified with the table descriptions of quality assessment values provided in Table 8. Table 8 below comprises a set of assessment values from 1 to Q and corresponding descriptions for each assessment value of the corresponding quality of implementation of recommendation.

TABLE 8

| Assessment | |
|---|---|
| value | Description of recommendation implementation quality |
| ? | An unexpected combination of functional state assessment values before and |
| | after training requires an in-depth study of the causes of this state after |
| | training |
| 5 | Nearly ideally matched load intensity during training |
| 4 | Well-matched load intensity during training |
| 3 | Insufficient load intensity during training |
| 2 | Excessive load intensity during training |
| 1 | Critically incorrect load intensity during training, risk of complications |

### Example 8

Example 8 is concerned a method of assessment and correction of the functional state of a patient who undergoes rehabilitation, implemented as follows.

The first ECG was recorded, after which the patient performed a therapeutic test. The therapeutic test consisted of a prescribed physical exercise 10 squats in 45 seconds, and a second ECG was immediately recorded.

The obtained ECGs were processed. For each of the two ECGs, the corresponding sets of V1 and V2 heart rate variability (HRV) parameters were calculated. The difference Δ between the corresponding parameters of the obtained V1 and V2 heart rate variability sets was calculated. A group G was formed consisting of the V1 set of HRV parameters, the V2 set of HRV parameters, and the calculated difference Δ between the parameter sets.

The resulting group of parameters was then processed in the patient functional assessment unit to obtain a functional assessment value. The obtained value was “5”.

This functional assessment value was compared with the tabular assessment values selected from Table 1 for the corresponding therapeutic test-physical exercise consisting of 10 squats in 45 seconds.

In this example, value “5” corresponds to the patient's functional state being “fully recovered”, i.e., a state of recovery with an increase in the initial level of functional capacity, characterized by an increase in the efficiency of work performed over a given period of time.

Recommendations were formulated for the athlete based on the determined compliance of the obtained functional state value “5” and the standard recommendation: “Perform exercises in the aerobic and developmental zones (HR 95-110% of the ATHR), but restorative and rehabilitative exercises are also permitted”.

In this example, 90-minute walks in the fresh air were recommended.

After receiving these recommendations, the patient went outside and took a 90-minute walk.

After returning from the walk, an ECG was recorded, after which the patient completed a therapeutic test. A similar therapeutic test, performed before the recommendation, was used: 10 squats in 45 seconds. ECG 4 was immediately recorded.

The resulting ECG3 and ECG4 were processed. The corresponding sets of V3 and V4 heart rate variability (HRV) parameters were calculated for each of the two recorded ECGs. The difference Δ between the corresponding parameters of the obtained V3 and V4 heart rate variability sets was calculated. A group G was formed consisting of the V3 HRV parameter set, the V4 HRV parameter set, and the calculated Δ difference between the parameter sets.

The resulting parameter group was then processed in the functional state assessment unit. The obtained value was “3”.

The obtained patient's functional state assessment values before and after following the recommendations were compared with the tabular values in Table 7 to obtain a quality assessment value of implementation of the recommendations. Table 7 (shown above) comprises a set of assessment values from 1 to Q and corresponding descriptions for each assessment value of the corresponding quality of implementation of recommendations provided in Table 8.

The obtained quality assessment value was determined to correspond to one of the assessment values from 1 to 6 provided in Table 8. Table 8 includes six quality descriptions selected from a group consisting of an unexpected combination of functional state assessments, a nearly ideally selected load intensity during training, a well-selected load intensity during training, insufficient load intensity during training, excessive load intensity during training, and a critically incorrect load intensity during training.

In this example, value “5” before following the recommendations and value “3” after following the recommendations correspond to “Nearly ideally matched load intensity during training”.

### Example 9

Example 9 is concerned a method of assessment and correction of the functional state of a top-class athlete (International Master of Sports in Judo).

The first ECG was recorded at rest before morning training. Then, the athlete performed the first therapeutic test. The therapeutic test consisted of a prescribed physical load of 30 squats in 45 seconds. Then the second ECG was immediately recorded.

The athlete then performed a second therapeutic test. The therapeutic test consisted of 30 forced inhalations and slow exhalations in 45 seconds. A third ECG was immediately recorded. The obtained ECG1, ECG2, and ECG3 were processed. The corresponding sets of heart rate variability (HRV) parameters V1, V2, and V3 were calculated for each of the three recorded ECGs.

The differences Δ12 and Δ13 between the corresponding parameters of the obtained sets of heart rate variability V1 and V2, V1 and V3 were calculated. Two groups of parameters were formed: one group consisting of the set of HRV parameters V1, the set of HRV parameters V2, and the calculated difference Δ12 between the parameter sets; and a parameter group consisting of the set of HRV parameters V1, the set of HRV parameters V3, and the calculated difference Δ13.

The resulting group was then processed in the functional state assessment unit to obtain the athlete's functional state assessment value. The resulted assessment value was “6” for the first pair of HRV parameter sets and the assessment value “5” for the second pair of HRV parameter sets. The obtained assessment values were compared with the tabular assessment values selected from Table 1 for the first therapeutic test (i.e., 30 squats in 45 seconds) and Table 2 for the second therapeutic test (i.e., 30 forced inhalations and slow exhalations). The corresponding text explanations for each assessment value for the corresponding functional state were also compared.

In this example, assessment values “6” and “5” correspond to the athlete's functional state: “supercompensation” and “good gas exchange compensation”. A description of the functional state and the corresponding standard recommendations are also provided in Table 1 and Table 2.

Recommendations were based on the identified correspondence between the obtained functional state values of “6” and “5” and the tabular assessment value and text explanation of the functional state, as well as on the standard recommendation: “Perform exercises in the anaerobic zone (HR 110-120% of the ATHR), but aerobic, restorative, and rehabilitative exercises are also permitted”, as well as “Perform physical activity: aerobic-anaerobic at the ATHR, low- and moderate-intensity aerobic”.

A general recommendation was issued to conduct sparring with elite athletes at maximum body stress, i.e., high-intensity physical activity.

Three electrocardiograms were recorded after doing exercises to obtain the quality assessment value of implementation of formed recommendations. After training, the fourth ECG4 was first recorded at rest, after which the athlete performed the third therapeutic test. The therapeutic test consisted of a regulated physical load of 30 squats in 45 seconds, and the fifth ECG5 was immediately recorded. After which the athlete performed the fourth therapeutic test. The therapeutic test consisted of 30 forced inhalations and slow exhalations for 45 seconds, and the sixth ECG6 was immediately recorded.

The obtained ECG4, ECG5, and ECG6 were processed. The corresponding sets of V4, V5, and V6 HRV parameters were calculated for each of the three recorded ECGs. The differences Δ45 and Δ46 between the corresponding parameters of the obtained sets of V4 and V5, and V4 and V6 heart rate variability were calculated. Two groups of parameters were formed: one group consisting of the set of V4 HRV parameters, the set of V5 HRV parameters, and difference Δ45 between the parameter sets; and a parameter group consisting of the set of V4 HRV parameters, the set of V6 HRV parameters, and difference Δ46.

The resulting group of parameters was processed in the functional state assessment unit to obtain the athlete's functional state assessment value. The obtained value was “2” for the third pair of HRV parameter sets and a value “3” was for the fourth pair of HRV parameter sets.

The resulting assessment value was compared with the tabular assessment values selected from Table 1 for the first therapeutic test, i.e., 30 squats in 45 seconds, and Table 2 for the second therapeutic test, i.e., 30 forced inhalations and slow exhalations. The compliance of each functional state assessment value was identified with the table descriptions of assessment values provided in Table 1 and Table 2 for the corresponding therapeutic tests.

In this example, assessment values of “3” and “2” correspond to the athlete's functional state: “overstrain and insufficient compensation of gas exchange”. The functional state description and corresponding recommendations are provided in Table 1 and Table 2.

Recommendations to the athlete were based on the identified correspondence between the obtained functional state values “2” and “3” and the tabular assessment values, as well as the text descriptions of the functional state provided in Tables 1 and 2. Standard recommendation is: “Perform exercises in the recovery or aerobic zones (HR 70-80% of the ATHR), but rehabilitative exercise is permitted”, as well as “Perform low-intensity aerobic physical activity”.

In the example described, a general recommendation was given: 45 seconds of exercise on an exercise bike.

A heart rate was measured during the exercise using a Garmin heart rate monitor to monitor the implementation of the recommendation.

The athlete's functional state was corrected by adjusted athlete's exercise process, that is, by increasing or decreasing pedaling speed. For this purpose, heart rate was measured using a Garmin heart rate monitor during exercise.

During an exercise, the heart rate measured using the heart rate monitor was higher than the recommended 145 beats per minute, so the trainer recommended reducing the pedaling speed.

### Example 10

Example 10 is concerned a method of assessment and correction of the functional state of a 7-year-old child with a sleep disorder.

The first ECG1 was recorded at rest, after which the child performed the first therapeutic test. The therapeutic test was a regulated physical load of 30 squats in 45 seconds, and a second ECG2 was immediately recorded. After which the child performed a second therapeutic test. The therapeutic test was 30 forced inhalations and slow exhalations for 45 seconds, and a third ECG3 was immediately recorded.

The obtained ECG1, ECG2, ECG3 were processed, for which the corresponding sets of V1, V2, V3 heart rate variability (HRV) parameters were calculated for each of the three recorded ECGs, the difference Δ12 and Δ13 between the corresponding parameters of the obtained sets V1 and V2, V1 and V3 heart rate variability was calculated, two groups of parameters were formed, one group consisting of the set V1 of HRV parameters, the set V2 of HRV parameters and the calculated difference Δ12 between the sets of parameters and a group of parameters consisting of the set V1 of HRV parameters, the set V3 of HRV parameters and the calculated difference Δ13.

The resulting group was then processed in the functional state assessment unit to obtain a functional state assessment value. This resulted in a value of “3” for the first pair of HRV parameter sets and a value of “4” for the second pair of HRV parameter sets.

The resulting assessment value was compared with the tabular assessment values selected from Table 1 for the first therapeutic test, i.e., 30 squats in 45 seconds, and Table 2 for the second therapeutic test, i.e., 30 forced inhalations and slow exhalations, along with the corresponding text explanations for each assessment value for the corresponding functional state.

The obtained functional state assessment values were compared to one of the assessment values provided in the tables for the corresponding therapeutic tests before implementing the recommendations.

In this example, assessment values of “3” and “4” correspond to the child's functional state: “fatigue and good gas exchange compensation”. The functional state description and corresponding standard recommendations are provided in Table 1 and Table 2.

Recommendations for the child were based on the identified correspondence of the obtained functional state values “3” and “4” with the tabular assessment values, as well as the text explanations of the functional state provided in Tables 1 and 2. Standard recommendation was: “Perform exercises exclusively in the aerobic zone (HR 80-95% of the ATHR), but restorative and rehabilitative exercise is also permitted”, as well as “Perform low- and moderate-intensity aerobic physical activity”.

In the example described, a walk in the fresh air before bedtime for at least 30 minutes was recommended. After receiving these recommendations, the child went outside before bedtime and took a 35-minute walk.

After returning from the walk, ECG 4 was recorded. The resulting ECG 4 was processed, which involved calculating the corresponding V4 sets of heart rate variability (HRV) parameters for the recorded ECG, calculating the difference Δ14 between the corresponding parameters of the obtained V1 and V4 sets, and forming a parameter group consisting of the V1 set of HRV parameters, the V4 set of HRV parameters, and the calculated Δ14 difference between the parameter sets.

The resulting parameter group was then processed in the functional state assessment unit to obtain the assessment value for the child's functional state after following the recommendations. This resulted in a value of “4” for the third pair of HRV parameter sets. The obtained assessment value before following the recommendations and the obtained assessment value after following the recommendations were compared with the tabular assessment values selected from Table 7 for assessing the quality of the training session, along with the corresponding text explanation for the assessment value for the corresponding functional state.

The obtained functional state assessment value was compared with one of the assessment values provided in the corresponding table describing the quality of recommendation implementation.

In this example, an assessment value of “4” corresponded to the description of the quality of recommendation implementation: “Well-matched load intensity during training”. The description of the quality of recommendation implementation is provided in Table 8.

Then the child went to bed. It was recommended to conduct a functional state assessment upon awakening. For this purpose, an ECG (5) was recorded after waking. The child then performed a third therapeutic test similar to the first therapeutic test, namely, 30 squats for 45 seconds, after which an ECG (6) was recorded. The obtained ECG5 and ECG6 were processed.

For each of the two recorded ECG5 and ECG6, the V5 and V6 heart rate variability (HRV) parameters were calculated. The difference Δ56 between the corresponding parameters of the obtained V5 and V6 heart rate variability sets was calculated. A parameter group was formed consisting of the V5 HRV parameter set, the V6 HRV parameter set, and the calculated Δ56 difference between the parameter sets.

The resulting parameter group was then processed in the functional state assessment unit, using one of the mathematical models described above, to obtain a functional state assessment value for the child. This resulted in a functional state assessment value of “4”.

The resulting functional state assessment value was compared with the tabular assessment values selected from Table 1 for the corresponding therapeutic test-physical exercise consisting of 30 squats in 45 seconds.

Table 1 comprises a set of values for the child's functional state, ranging from 1 to M=6, along with text explanations for each value and standard recommendations. Table 1 includes descriptions of six states selected from a group consisting of critical overstrain, overstrain, fatigue, incomplete recovery, complete recovery, and supercompensation.

In this example, value “4”, according to Table 1, corresponds to the child's functional state of “incomplete recovery”, i.e., a state arising as a result of activity and by a temporary decrease in the body's functional capacity, characterized by a decrease in the effectiveness of work performed over a certain period of time. The obtained value of “4” for the child's functional state assessment was determined to correspond to one of the assessment values provided in Table 1.

Recommendations for the child were formed based on the determined correspondence between the obtained value of “4” and the standard recommendation: “Perform exercises in the aerobic zone with a small percentage of time spent in the developmental zone (HR 90-100% of the ATHR), but restorative and rehabilitative exercise is also permitted”.

In this example, it was recommended to continue taking walks in the fresh air before bedtime for at least 30 minutes. To monitor compliance with the recommendations, heart rate was measured during the implementation of the recommendations.

Functional state was corrected through adjusted implementation of the recommendations, i.e., increasing or decreasing the walking speed during a walk in the fresh air. For this purpose, the heart rate was measured during the walk using a Polar H10 heart rate monitor.

During the walk, the child's heart rate did not rise to the recommended 60-70% of the normal heart rate, so the child increased his walking speed.

As a result, the parents noted an improvement in the child's sleep quality.

A method for assessing and correcting a person's functional state, according to a third embodiment of the invention, is illustrated by Example 11.

### Example 11

Example 11 is concerned a method of assessment and correction of an athlete's functional state, implemented as follows.

A first ECG1 was recorded, after which the athlete performed a therapeutic test. The therapeutic test consisted of 20 push-ups in 45 seconds. A second ECG2, was recorded after completing the push-ups.

A Fourier transform was performed on the recorded ECGs to obtain a Fourier amplitude spectrum for each recorded ECG. The transformed ECG1 and ECG2 were processed, i.e., each obtained Fourier amplitude spectrum was discretized to obtain a discrete form of Fourier amplitude spectrum (DFECG) for each transformed ECG. For the resulting pair of DFECG, the difference Δ DFECG between the selected pair of discrete Fourier amplitude spectra was calculated, and a parameter group consisting of the DFECG pair and their calculated difference Δ DFECG was formed.

The resulting parameter group was processed in the functional state assessment unit. As a result, a functional state assessment value of “1” was obtained.

The obtained functional state assessment value for the athlete was compared with the tabular assessment values selected from Table 1 for the corresponding therapeutic test, i.e., 20 push-ups in 45 seconds. Table 1 comprises a set of functional state assessment values from 1 to M=6, along with corresponding text descriptions for each assessment value for the corresponding functional state, and standard recommendations.

The athlete's functional state assessment was compared to one of the assessment values from 1 to M=6, as shown in Table 1. Table 1 comprises descriptions of six states selected from the group consisting of critical overstrain, overstrain, fatigue, incomplete recovery, complete recovery, and supercompensation.

In this example, a value of “1” corresponds to the individual's functional state: significant (critical) overstrain. A description of the functional state and the corresponding standard recommendations are provided in Table 1.

Recommendations were formulated based on the identification of compliance of the obtained functional state value of “1” and the assessment values provided in the table, the text descriptions of the functional state, and the standard recommendation, i.e., a complete ban on physical activity and an urgent health assessment. To monitor compliance with the recommendations, the sports medicine physician called an ambulance team to conduct a health assessment of the athlete.

The athlete's functional state was corrected through controlled implementation of the recommendations, i.e., “Complete prohibition of physical activity”. For this purpose, the athlete was taken on a gurney and forwarded to an ambulance.

While in the ambulance, an acute inflammatory process was diagnosed.

### INDUSTRIAL APPLICABILITY

The method enables monitoring, assessment, and correction of a person's functional state. The method can be used in various settings, including hospitals, first-aid stations, as well as for the correction of a person's functional state outside of healthcare facilities in sports organizations, fitness clubs, gyms, resorts, schools and kindergartens, with personal support, in clinical practice for the assessment and correction of a functional state taking into account the presence of a chronic and/or acute pathological process, as well as for monitoring a person undergoing recovery and/or rehabilitation.

1. A method for assessment and correction of a person's functional state, the method comprising the following steps:
recording two electrocardiograms (ECGs) of at least a first lead, wherein the first ECG1 is recorded initially, and the second ECG2 is recorded after the person has completed a therapeutic test;
calculating corresponding sets of heart rate variability (HRV) parameters V1 and V2 for the recorded ECG1 and ECG2;
calculating a difference Δ between the obtained sets of heart rate variability parameters V1 and V2;
forming a group G of parameters consisting of the set of HRV parameters V1, the set of HRV parameters V2, and the calculated difference Δ between the sets of parameters;
processing the formed group G of parameters consisting of V1, V2, and A in a unit for assessing the person's functional state using a mathematical model; said mathematical model is selected from a group consisting of a decision tree, a neural network, and one of three regressions: linear, logistic, and probit, to obtain the value E of the person's functional state assessment;
comparing the obtained value of the person's functional state assessment with the tabular assessment values selected from the table for the corresponding therapeutic test, wherein the table for the corresponding therapeutic test comprises a set of functional state assessment values from 1 to M and descriptions for each assessment value of the corresponding functional state, as well as standard recommendations; and identifying the compliance of the obtained functional state assessment value to one of the assessment values provided in the table of the corresponding therapeutic test;
forming recommendations for the person based on the functional state descriptions and standard recommendations provided in the table for the identified functional state assessment value;
monitoring the implementation of the formed recommendations and adjusting the implementation process based on the monitoring data;
implementing the correction of the functional state by following the formed recommendations by the person.

2. The method of claim 1, wherein the step of monitoring the implementation of formed recommendations is performed by taking at least one measurement selected from the group consisting of heart rate, palpation, percussion, auscultation, blood pressure, temperature, blood saturation, blood lactate accumulation level, spirometry, and the Minor test during the implementation of recommendations, or by recording cardiographic data or biometric data during the implementation of recommendations.

3. The method of claim 1, wherein the therapeutic test comprises at least one exercise selected from the group consisting of physical exercise, functional load tests, respiratory exercise, physiotherapeutic exercise, psychoemotional practice, and/or at least one influence selected from the group consisting of medication administration, physiotherapeutic influence, psychoemotional influence, respiratory influence, or combinations thereof.

4. The method of claim 1, wherein the descriptions in the table for functional state assessment value for each corresponding functional state are selected from the group consisting of text explanations, videos, images, video instructions, diagrams, animations, or combinations thereof.

5. The method of claim 1, further comprising the step of identification of compliance of the obtained person's functional state assessment value to one of the assessment values from 1 to M=6, provided in the table and including six states.

6. The method of claim 3, wherein when using the therapeutic test from the group consisting of physical exercise, functional load test, physiotherapeutic exercise, and psychoemotional practice, or combinations thereof,
providing an identification of compliance of the obtained person's functional state assessment value to one of the assessment values from 1 to M=6, provided in the table and comprising six states selected from the group consisting of critical overstrain, overstrain, fatigue, incomplete recovery, complete recovery, and supercompensation.

7. The method of claim 3, wherein when using respiratory exercise,
providing an identification of compliance of the obtained person's functional state assessment value to one of the assessment values from 1 to M=6, provided in the table and comprising six states selected from the group consisting of a pathological condition, overstrain of the gas exchange systems, insufficient compensation of gas exchange, moderate compensation of gas exchange, good compensation of gas exchange, and supercompensation.

8. The method of claim 1, comprising the step of identification of compliance of the obtained person's functional state assessment value to one of the assessment values from 1 to M=5, provided in the table and comprising five states.

9. The method of claim 3, wherein when using the therapeutic test from the group consisting of taking medications, physiotherapeutic influence, psychoemotional influence, respiratory influence, or combinations thereof,
providing an identification of compliance of the obtained person's functional state assessment value to one of the assessment values from 1 to M=5, provided in the table and comprising five states selected from the group consisting of favorable improvement, unfavorable improvement, favorable deterioration, unfavorable deterioration, and unchanged state.

10. The method of claim 1, comprising the step of identification of compliance of the obtained person's functional state assessment value to one of the assessment values from 1 to M=4, provided in the table and comprising four states.

11. The method of claim 3, comprising the step of identification of compliance of the obtained person's functional state assessment value to one of the assessment values from 1 to M=4, provided in the table and comprising four states selected from the group consisting of overstrain, fatigue, recovery, and supercompensation.

12. The method of claim 2, wherein further obtaining person's functional state assessment values in the range from 0 to M=10.

13. The method of claim 12, wherein the value 0 corresponds to the person's worst functional state and the value 10 corresponds to the person's best functional state.

14. A method for assessment and correction of a person's functional state, the method comprising the following steps:
recording at least three electrocardiograms (ECGs) of at least a first lead, wherein the first ECG1 is recorded initially, the second ECG2 is recorded after the person has completed a therapeutic test, and the third ECG3 and subsequent ECGs are recorded after the person has completed the second and subsequent therapeutic tests, respectively;
selecting at least one pair of recorded electrocardiograms ECGi and ECGj, where said indices i and j are selected from the range from 1 to N, where N is the number of recorded ECGs, wherein i≠j;
calculating corresponding sets Vi and Vj of heart rate variability (HRV) parameters for each ECG from the selected at least one pair of ECGi and ECGj;
calculating a difference Δij between each calculated at least one pair of sets Vi and Vj of the HRV parameters;
forming a group Gij of parameters for each calculated at least one pair of sets of Vi and Vj of the HRV parameters, consisting of the set of Vi of the HRV parameters, the set of Vj of the HRV parameters, and their calculated difference Δij,
processing each of the formed group Gij of parameters in a unit for assessing the person's functional state using a mathematical model; said mathematical model is selected from a group consisting of a decision tree, a neural network, and one of three regressions: linear, logistic, and probit, to obtain the value Eij of the person's functional state assessment for each formed group Gij of parameters;
comparing each obtained person's functional state assessment value Eij of the corresponding selected pair of recorded ECGi and ECGj with the tabular assessment values selected from the table for the corresponding therapeutic test, wherein the table for the corresponding therapeutic test comprises a set of functional state assessment values from 1 to M and descriptions for each assessment value of the corresponding functional state, as well as standard recommendations; and identifying the compliance of the obtained functional state assessment value Eij to one of the assessment values provided in the table of the corresponding therapeutic test;
forming recommendations for the person based on the functional state descriptions and standard recommendations provided in the table for the identified functional state assessment value for all pairs of recorded ECGs;
monitoring the implementation of the formed recommendations and adjusting the implementation process based on the monitoring data;
implementing the correction of the functional state by following the formed recommendations by the person.

15. The method of claim 14, further comprising a step of obtaining a quality assessment value of implementation of the formed recommendations, by performing the following steps:
recording at least two electrocardiograms (ECGs) of at least a first lead; calculating corresponding heart rate variability (HRV) parameters; calculating the difference Δ between the HRV parameter; forming and processing a group G of parameters; and obtaining a person's functional state assessment value E before implementation of recommendations; then forming and implementing recommendations by implementation the method steps of claim 14;
recording at least one additional ECG, wherein a therapeutic test is performed by the person between each successive pair of additional ECGs;
processing the additional ECGs and obtaining a person's functional state assessment value after implementation of the recommendations;
comparing the obtained value before receiving the recommendations and the value after implementation of recommendations with the values provided in a table of quality assessment values, to obtain the quality assessment value of implementation of recommendations, wherein said table of quality assessment values comprises the assessment value before performing the recommendations, the assessment value after implementation of recommendations, and the corresponding quality assessment value of implementation of recommendations.

16. The method of claim 15, further comprising
identifying the compliance of the obtained quality assessment value of implementation of recommendations with the table descriptions of quality assessment values, wherein the table descriptions of quality assessment values comprises a quality assessment values from 1 to Q and corresponding descriptions for each assessment value,
wherein the descriptions in the table descriptions of quality assessment values are selected from the group consisting of text explanations, videos, images, video instructions, diagrams, animations, or combinations thereof.

17. A method for assessment and correction of a person's functional state, the method comprising the following steps:
recording at least two electrocardiograms (ECGs) of at least a first lead, wherein the first ECG1 is recorded initially, the second ECG2 is recorded after the person has completed a therapeutic test, and each subsequent ECGs is recorded after the person has completed subsequent therapeutic tests;
selecting at least one pair of recorded electrocardiograms ECGi and ECGj, where said indices i and j are selected from the range from 1 to N, where N is the number of recorded ECGs, wherein i≠j;
performing a Fourier transform for each ECG from the selected at least one pair of ECGi and ECGj to obtain a Fourier amplitude spectrum for each recorded ECG, and discretizing each obtained Fourier amplitude spectrum to obtain a discrete form of the Fourier amplitude spectrum (DFECG) for each ECG from the selected at least one pair of ECGi and ECGj;
calculating a difference Δ DFECGij between the discrete types of the Fourier amplitude spectrum DFECGi and DFECGj for each selected at least one pair of ECGi and ECGj;
forming a group Gij of parameters for each selected at least one pair of sets of Vi and Vj of the HRV parameters, consisting of the set of Vi of the HRV parameters, the set of Vj of the HRV parameters, and their calculated difference Δ DFECGij;
processing the formed group Gij of parameters in a unit for assessing the person's functional state using a mathematical model; said mathematical model is selected from a group consisting of a decision tree, a neural network, and one of three regressions: linear, logistic, and probit, to obtain the person's functional state assessment value Eij for each formed group Gij;
comparing each obtained person's functional state assessment value Eij of the corresponding selected pair of recorded ECGi and ECGj with the tabular assessment values selected from the table for the corresponding therapeutic test, wherein the table for the corresponding therapeutic test comprises a set of functional state assessment values from 1 to M and descriptions for each assessment value of the corresponding functional state, as well as standard recommendations; and identifying the compliance of the obtained functional state assessment value Eij to one of the assessment values provided in the table of the corresponding therapeutic test;
forming recommendations for the person based on the functional state descriptions and standard recommendations provided in the table for the identified functional state assessment value for all selected pairs of the recorded ECGs;
monitoring the implementation of the formed recommendations and adjusting the implementation process based on the monitoring data;
implementing the correction of the functional state by following the formed recommendations by the person.

18. A system for assessment and correction of a person's functional state value, comprising
a examination scenario control unit, connected to an electrocardiograph configured to record a series of electrocardiograms (ECG) of at least a first lead before and after the person has implemented at least one therapeutic test, and connected to an ECG accumulation unit, which in turn is connected to the electrocardiograph, and
connected in series:
a unit for calculation sets of characteristic parameters for each of the recorded ECGs, connected to the ECG accumulation unit;
a unit for calculation a difference Δ between pairs of the sets of characteristic parameters and for forming a group of parameters, said group consists of two sets of characteristic parameters and the calculated difference 4;
a unit for assessing person's functional state value, configured to process the formed group of parameters and obtain a person's functional state assessment value by using a mathematical model selected from a group consisting of a decision tree, a neural network, one of three regressions—linear, logistic, and probit;
a unit for selection a description of a functional state and standard recommendations from a built-in table for corresponding therapeutic test and forming standard recommendations based on the obtained assessment value, wherein the table comprises a set of functional state assessment values from 1 to M and descriptions of the corresponding functional state and standard recommendations for each assessment value;
a unit for forming and storing recommendations;
a unit for accumulation monitoring data, configured to accumulate the monitoring results of fulfilment the formed recommendations, and further to detect deviations from implementation of recommendations;
a unit for forming instructions for correction an implementation of formed recommendations for correction the person's functional state.

19. The system of claim 18, further comprising a unit for inputting monitoring data, connected to the unit for accumulation monitoring data.

20. The system of claim 18, further comprising a unit for updating formed recommendations, connected to the unit for forming and storing recommendations.
